(19) United States
(12) Patent Application Publication
(10) Pub. No.: US 2010/0137353 A1
(43) Pub. Date: Jun. 3, 2010

(54) TRICYCLIC COMPOUNDS AS ANTIBACTERIALS

(75) Inventor: Neil David Pearson, Collegeville, PA (US)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 12/596,695

(22) PCT Filed: Apr. 17, 2008
(86) PCT No.: PCT/EP2008/054667

§ 371 Date: Oct. 20, 2009

(30) Foreign Application Priority Data
Apr. 20, 2007 (GB) 0707706.8

(51) Int. Cl. A61K 31/4375; C07D 471/16; C07D 471/06; A61K 31/437; A61P 31/04; A61P 31/06

(52) U.S. Cl. 514/292; 546/86; 546/94; 514/294

(57) ABSTRACT

Tricyclic nitrogen containing compounds and their use as antibacterials.

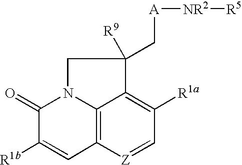

This invention relates to novel compounds, compositions containing them and their use as antibacterials, including use in the treatment of tuberculosis. WO9937635, WO0021948, WO0021952, WO0043383, WO0078748, WO0107432, WO0107433, WO0250040, WO02/08224, WO02/24684 WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO2006002047, WO2006014580, WO2006010040, WO2006017326, WO2006012396, WO2006017468, WO2006020561, WO2006081179, WO2006081264, WO2006081289, WO2006081178, WO2006081182, WO01/25227, WO02/40474, WO02/07572, WO2004024712, WO2004024713, WO2004035569, WO2004087647, WO2004089947, WO2005016916, WO2005097781, WO2006010831, WO2006021448, WO2006032466, WO2006038172, WO2006046552, WO06099884, WO06126171, WO06137485, WO06105289, WO06125974, WO06134378, WO07016610, WO07081597, WO07071936, WO07115947, WO07118130, WO07122258, WO08006648, WO08003690 and WO08009700 disclose quinoline, naphthyridine, morpholine, cyclohexane, piperidine and piperazine derivatives having antibacterial activity. WO2004104000 discloses tricyclic condensed ring compounds capable of selectively acting on cannabinoid receptors.

This invention provides a compound of formula (I) or a pharmaceutically acceptable salt thereof:

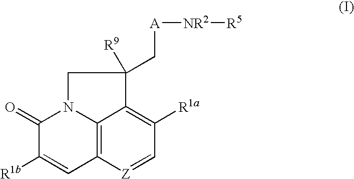

wherein:
Z is CH or N;
R^{1a} and R^{1b} are independently selected from hydrogen; halogen; cyano; (C₁₋₆)alkyl; (C₁₋₆)alkylthio; trifluoromethyl; trifluoromethoxy; carboxy ; hydroxy optionally substituted with (C₁₋₆)alkyl or (C₁₋₆)alkoxy-substituted(C₁₋₆)alkyl; (C₁₋₆)alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl;
R² is hydrogen, or (C₁₋₄)alkyl, or together with R⁶ forms Y as defined below;
A is a group (ia) or (ib):

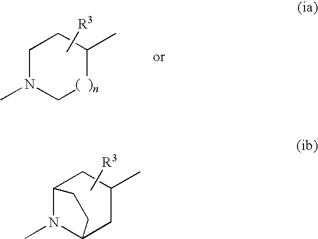

in which: R³ is as defined for R^{1a} or R^{1b} or is oxo and n is 1 or 2:
or A is a group (ii)

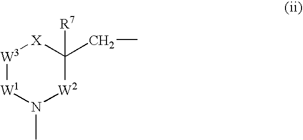

W¹, W² and W³ are CR⁴R⁸
or W² and W³ are CR⁴R⁸ and W¹ represents a bond between W³ and N.
X is O, CR⁴R⁸, or NR⁶;
one R⁴ is as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆)alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
R⁵ is a group —X^{1a}—X^{2a}—X^{3a}—X^{4a} in which:
   X^{1a} is CH₂, CO or SO₂;
   X^{2a} is CR^{14a}R^{15a};
   X^{3a} is NR^{13a}, O, S, SO₂ or CR^{14a}R^{15a}; wherein:
   each of R^{14a} and R^{15a} is independently selected from: H; (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; aryl; aryl(C₁₋₄)alkyl; aryl(C₁₋₄)alkoxy; provided that R^{14a} and R^{15a} on the same carbon atom are not both selected from optionally substituted hydroxy and optionally substituted amino; or

R^{14a} and R^{15a} together represent oxo;

R^{13a} is hydrogen; trifluoromethyl; (C₁₋₆)alkyl; (C₂₋₆)alkenyl; (C₁₋₆)alkoxycarbonyl; (C₁₋₆)alkylcarbonyl; or aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl; or

two R^{14a} groups or an R^{13a} and an R^{14a} group on adjacent atoms together represent a bond and the remaining R^{13a}, R^{14a} and R^{15a} groups are as above defined; or
two R^{14a} groups and two R^{15a} groups on adjacent atoms together represent bonds such that X^{2a} and X^{3a} is triple bonded;

X^{4a} is phenyl or C or N linked monocyclic aromatic 5- or 6-membered heterocycle containing up to four heteroatoms selected from O, S and N and: optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C ₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; aryl, aryl(C₁₋₄)alkyl or aryl(C₁₋₄)alkoxy; and

optionally N substituted by trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl(C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl; and

R⁹ is hydrogen or hydroxy, or when Z is N R⁹ may instead be fluoro.

This invention also provides a method of treatment of bacterial infections including tuberculosis in mammals, particularly in man, which method comprises the administration to a mammal in need of such treatment an effective amount of a compound of formula (I), or a pharmaceutically acceptable salt thereof.

The invention also provides the use of a compound of formula (I), or a pharmaceutically acceptable salt thereof, in the manufacture of a medicament for use in the treatment of bacterial infections including tuberculosis in mammals.

The invention also provides a pharmaceutical composition comprising a compound of formula (I), or a pharmaceutically acceptable salt thereof, and a pharmaceutically acceptable carrier.

In a particular aspect each R^{1a} and R^{1b} is independently hydrogen, (C₁₋₄)alkoxy, (C₁₋₄)alkylthio, (C₁₋₄)alkyl, cyano, carboxy, hydroxymethyl or halogen; more particularly hydrogen, methoxy, methyl, cyano, or halogen.

In certain embodiments each R^{1a} and R^{1b} is hydrogen, methoxy, methyl, or halogen, such as chloro or fluoro. In some embodiments only one group R^{1a} or R^{1b} is other than hydrogen. In particular embodiments Z is CH and R^{1a} is methoxy, fluoro or cyano and R^{1b} is hydrogen, more particularly R^{1a} is fluoro and R^{1b} is hydrogen. In other particular embodiments Z is N and R^{1a} is chloro, fluoro or methoxy. In further particular embodiments Z is N and both R^{1a} and R^{1b} are other than hydrogen, more particularly halogen, such as R^{1a} fluoro and R^{1b} chloro or fluoro.

In a particular aspect R² is hydrogen.

In a particular aspect Z is CH and R⁹ is hydrogen, and more particularly the stereochemistry at the carbon atom to which the group R⁹ is attached is R when R⁹ is H. In a further aspect Z is N and R⁹ is OH.

In a particular aspect, Z is N and the stereochemistry at the carbon atom to which the group R⁹ is attached is S.

Particular examples of R³ include hydrogen; optionally substituted hydroxy; optionally substituted amino; halogen; (C₁₋₄) alkyl; 1-hydroxy-(C₁₋₄) alkyl; optionally substituted aminocarbonyl. More particular R³ groups are hydrogen; CONH₂; 1-hydroxyalkyl e.g. CH₂OH; optionally substituted hydroxy e.g. methoxy; optionally substituted amino; and halogen, in particular fluoro. Most particularly R³ is hydrogen, hydroxy or fluoro.

In a particular aspect, when A is (ia), n is 1. In a further aspect R³ is in the 3- or 4-position. In a more particular aspect, A is (ia), n is 1 and R³ is in the 3-position, and more particularly is cis to the NR² group.

In particular embodiments, A is a group (ia) in which n is 1 and R³ is hydrogen or hydroxy. More particularly, where A is 3-hydroxy-piperidin-4-yl the configuration is (3R,4S)) or (3S,4R). Still more particularly where A is 3-hydroxy-piperidin-4-yl the configuration is (3R,4S).

In an alternative more particular aspect, when A is (ia), n is 1, R³ is in the 4-position and is methyl.

In a particular aspect, when A is (ii), X is CR⁴R⁸, R⁸ is H and R⁴ is H or OH and more particularly OH is trans to R7. In a further aspect W¹ is a bond. In another aspect R⁷ is H. In particular embodiments W¹ is a bond, X, W² and W³ are each CH₂ and R⁷ is H. Where A is 4-hydroxypyrrolidin-3-ylmethyl, in a particular aspect the configuration is (3S,4S). Where A is pyrrolidin-3-ylmethyl, in a particular aspect the configuration is 3S.

In a particular aspect, when A is (ii), X is O, R⁷ is H and W¹, W² and W³ are each CH₂.
X^{1a} is preferably CH₂.

X^{2a} is preferably CH₂ or together with X^{3a} forms a CH═CH or CC group.

X^{3a} is preferably CH₂, O, S or NH, or together with X^{2a} forms a CH═CH or C≡C group.

Preferred linker groups —X^{1a}—X^{2a}—X^{3a}— included —(CH₂)₂—O—, —CH₂—CH═CH—, —(CH₂)₃—, —(CH₂)₂—NH— or —CH₂CONH—.

Monocyclic aromatic heterocyclic groups for X^{4a} include pyridyl, pyrazinyl, pyrimidinyl, triazolyl, tetrazolyl, thienyl, isoimidazolyl, thiazolyl, furanyl and imidazolyl, 2H-pyridazone, 1H-pyrid-2-one. Preferred aromatic heterocyclic groups include pyrid-2-yl, pyrid-3-yl, thiazole-2-yl, pyrimidin-2-yl, pyrimidin-5-yl and fur-2-yl.

Preferred substituents on heterocyclic X^{4a} include halo especially fluoro, trifluoromethyl and nitro.

Preferred substituents on phenyl X^{4a} include halo, especially fluoro, nitro, cyano, trifluoromethyl, methyl, methoxycarbonyl, 1-methylethyl and methylcarbonylamino.

Preferably X^{4a} is pyrid-2-yl, fur-2-yl, 4-(1-methylethyl)phenyl, pyrid-3-yl, 2,5-difluorophenyl, 3-fluorophenyl, 5-fluoropyrid-3-yl, 3,5-difluorophenyl or thiazol-2-yl.

When used herein, the term “alkyl” includes groups having straight and branched chains, for instance, and as appropriate, methyl, ethyl, n-propyl, iso-propyl, n-butyl, iso-butyl, sec-butyl, t-butyl, pentyl and hexyl. The term ‘alkenyl’ should be interpreted accordingly.

Halo or halogen includes fluoro, chloro, bromo and iodo.

Haloalkyl moieties include 1-3 halogen atoms.

Compounds within the invention contain a heterocyclyl group and may occur in two or more tautomeric forms depending on the nature of the heterocyclyl group; all such tautomeric forms are included within the scope of the invention.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Compounds of the invention in which Z is N may also form N-oxides. Particular compounds according to the invention include those mentioned in the examples and their pharmaceutically acceptable salts or N-oxides.

Furthermore, it will be understood that phrases such as “a compound of formula (I) or a pharmaceutically acceptable salt thereof” are intended to encompass the compound of formula (I), a pharmaceutically acceptable salt of the compound of formula (I), an N-oxide of formula (I), a solvate of formula (I), or any pharmaceutically acceptable combination of these. Thus by way of non-limiting example used here for illustrative purpose, “a compound of formula (I) or a pharmaceutically acceptable salt” may include a pharmaceutically acceptable salt and/or N-oxide of a compound of formula (I) that is further present as a solvate.

Since the compounds of formula (I) are intended for use in pharmaceutical compositions it will readily be understood that in particular embodiments they are provided in substantially pure form, for example at least 60% pure, more suitably at least 75% pure and particularly at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and more particularly from 10% of a compound of the formula (I) or pharmaceutically acceptable salt and/or solvate thereof.

Particular compounds according to the invention include those mentioned in the examples and their pharmaceutically acceptable salts and solvates.

Pharmaceutically acceptable salts of the above-mentioned compounds of formula (I) include the acid addition or quaternary ammonium salts, for example their salts with mineral acids e.g. hydrochloric, hydrobromic, sulphuric nitric or phosphoric acids, or organic acids, e.g. acetic, fumaric ((2E)-2-butenedioic), succinic, maleic, citric, benzoic, p-toluenesulphonic (4-methylbenzene sulphonic), methanesulphonic, naphthalenesulphonic acid or tartaric acids. Compounds of formula (I) where Z is N may also be prepared as the N-oxide. The invention extends to all such derivatives.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes enantiomers and diastereoisomers at the attachment points of NR², R³ and/or R⁹. The different isomeric forms may be separated or resolved one from the other by conventional methods, or any given isomer may be obtained by conventional synthetic methods or by stereospecific or asymmetric syntheses. Certain compounds of formula (I) may also exist in polymorphic forms and the invention includes such polymorphic forms.

In a further aspect of the invention there is provided a process for preparing compounds of formula (I) in which R⁹ is H, and pharmaceutically acceptable salts thereof, which process comprises cyclising a compound of formula (IIA):

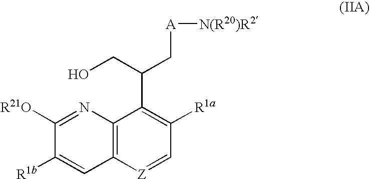

in which R²¹ is (C₁₋₆)alkyl such as methyl, R²⁰ is R⁵ or a group convertible thereto and R^{2′} is R² or a group convertible thereto, wherein Z, A, R^{1a}, R^{1b}, R² and R⁵ are as defined in formula (I), to give a compound of formula (IIB):

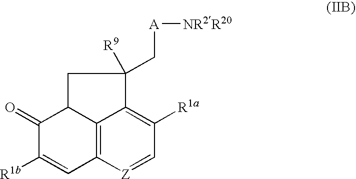

in which R⁹ is H, and and thereafter optionally or as necessary converting R²⁰ and R^{2′} to R⁵ and R², interconverting any variable groups, and/or forming a pharmaceutically acceptable salt thereof.

The cyclisation reaction is effected by treatment of the compound of formula (IIA) with an activating agent such as methanesulphonyl chloride, p-toluenesulphonyl chloride, methanesulfonic anhydride or p-toluene sulfonic anhydride and an organic base such as triethylamine or diisopropylethylamine. Mesylate or tosylate preparation takes place under standard conditions and the compound of formula (IIB) forms in situ.

In a further aspect of the invention there is provided a process for preparing compounds of formula (I) in which Z is CH and R⁹ is OH, and pharmaceutically acceptable salts thereof, which process comprises cyclising a compound of formula (IIC):

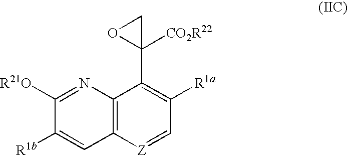

in which R²¹ is (C₁₋₆)alkyl such as methyl R²² is H or (C₁₋₆)alkyl such as methyl and R^{1a}, R^{1b} are as defined in formula (I), to give a compound of formula (IID):

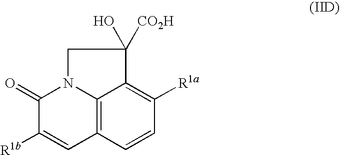

and and thereafter converting —CO₂H to —CH₂-A-NR²—R⁵, interconverting any variable groups, and/or forming a pharmaceutically acceptable salt thereof.

The cyclisation reaction may be effected by treatment of the compound of formula (IIC) with lithium perchlorate in acetonitrile or lithium hydroxide in water to give the tricyclic hydroxy-carboxylic acid (IID). Conversion of —CO₂H to —CH₂-A-NR²—R⁵ may be effected by methylation using methanol in sulphuric acid, followed by reduction to the diol with sodium borohydride in methanol, and conversion to the tosyl derivative with tosyl chloride/dibutyltin oxide. Reaction with amine HN-A-NR²⁰R^{2′} R²⁰ where R²⁰ is R⁵ or a group convertible thereto and R^{2′} is R² or a group convertible thereto, gives a compound of formula (IIB) in which R⁹ is OH.

Conveniently one of R²⁰ and R^{2′} is an N-protecting group, such as such as t-butoxycarbonyl, benzyloxycarbonyl or 9-fluorenylmethyloxycarbonyl. This may be removed by several methods well known to those skilled in the art (for examples see “Protective Groups in Organic Synthesis, T. W. Greene and P. G. M. Wuts, Wiley-Interscience, 1999), for example conventional acid hydrolysis with, for example trifluoroacetic acid or hydrochloric acid.

The free amine of formula (IIB) in which R²⁰ is hydrogen may be converted to NR²R⁵ by conventional means such as amide or sulphonamide formation with an acyl derivative, for compounds where X^{1a} is CO or SO₂ or, where X^{1a} is CH₂, by alkylation with an alkyl halide in the presence of base, acylation/reduction with an acyl derivative or reductive alkylation with an aldehyde under conventional conditions (see for examples Smith, M. B.; March, J. M. Advanced Organic Chemistry, Wiley-Interscience 2001). The compound of formula (IIA) may be prepared by the following Scheme 1:

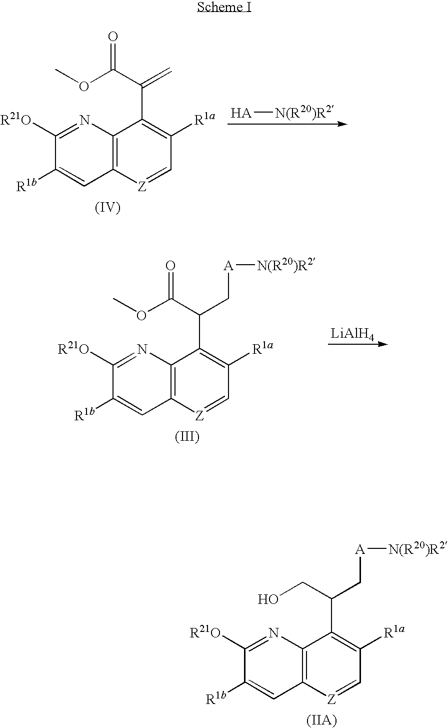

Compounds of general structure (III) may be prepared by reaction of acrylate ester (IV) with a compound HA-N(R²⁰)R^{2′}, such as a Boc protected amino-piperidine, under conventional conditions for Michael additions (see for examples Smith, M. B.; March, J. M. Advanced Organic Chemistry, Wiley-Interscience 2001). Reduction of (III) to (IIA) occurs upon treatment with lithium aluminium hydride under conventional conditions (see for examples Smith, M. B.; March, J. M. Advanced Organic Chemistry, Wiley-Interscience 2001).

The compound of formula (IIC) may be prepared by conventional epoxidation of the vinyl ester (IV) e.g. by oxidation with m-chloroperbenzoic acid or t-butyl hydrogen peroxide.

A route to intermediate (IV) where Z is CH is shown in Scheme 2a:

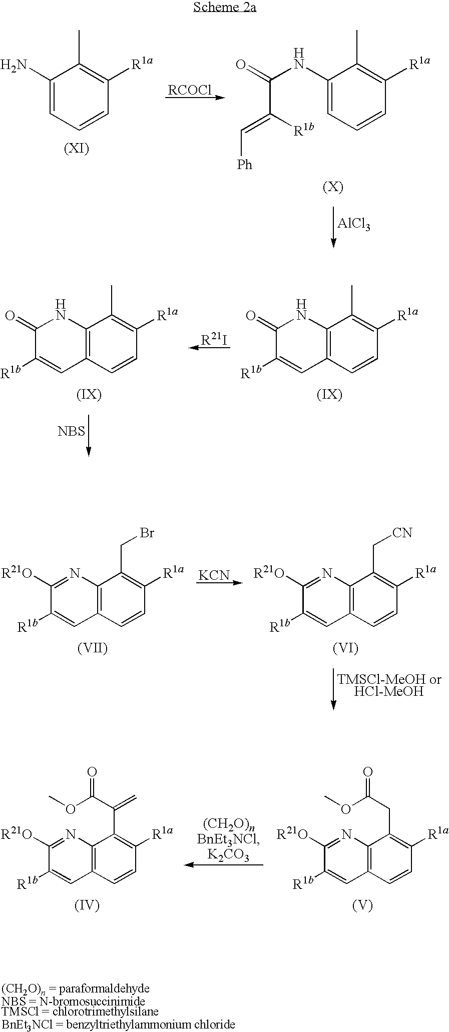

The aniline (XI) is converted to the cinnamide (X), which is cyclised with aluminium chloride (with loss of the phenyl moiety—See M. C. Elliot et al. S. R. Inglis et al. J. Med. Chem. 47 (22), 5405-5417 (2004)] Synlett, 5, 898-900 (2004)) to give (IX). This is selectively 0-alkylated with e.g. methyl iodide or dimethylsulphate to give (VIII) and the methyl group functionalised with N-bromosuccinimide to give the bromomethyl analogue (VII). This is converted to the nitrile (VI) by treatment with KCN, or with NaCN and tetrabutylammonium bromide, which undergoes acid-catalysed methanolysis (TMS-chloride or HCl in methanol) to the methyl ester (V), and then vinylation with paraformaldehyde. Some demethylated material is formed with (V), but this can be re-methylated with TMS-diazomethane. This route is particularly suitable for R^{1a}═F.

An alternative route to intermediate (IV) where Z is CH is shown in Scheme 3:

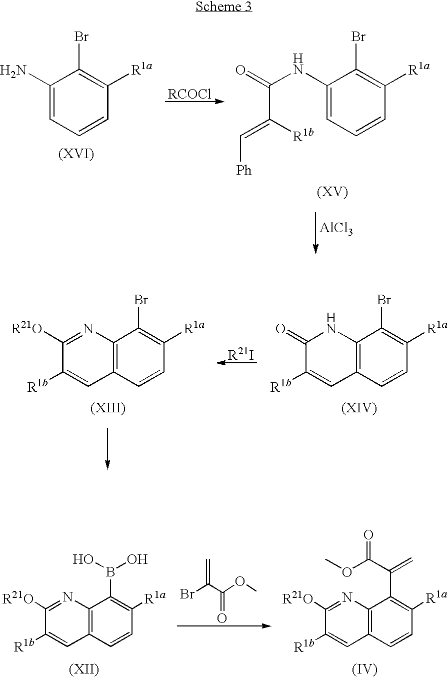

Quinolinone (XIV) may be prepared by reaction of commercially available aniline (XVI) with cinnamoyl chloride to give (XV) and its subsequent cyclisation (for an example of this procedure see Cottet, F.; Marull, M.; Lefebvre, O.; Schlosser, M European Journal of Organic Chemistry (2003), 8, 1559). (XIV) can be converted into the bromo-quinoline (XIII) under standard conditions (see for examples Smith, M. B.; March, J. M. Advanced Organic Chemistry, Wiley-Interscience 2001). The boronic acid (XII) can be synthesised from (XIII) under standard conditions (for an example see Li, W.; Nelson, D.; Jensen, M.; Hoerrner, R.; Cai, D.; Larsen, R.; Reider, P J. Org. Chem. (2002), 67(15), 5394). The coupling of (XII) with the known bromo-acrylate, (for synthesis see Rachon, J.; Goedken, V.; Walborsky, H. J. Org. Chem. (1989), 54(5), 1006) to give (IV) may be accomplished using a Suzuki coupling reaction (for conditions see Littke, A.; Dai, C.; Fu, G. J. Am. Chem. Soc. (2000), 122(17), 4020 This route is particularly suitable for R^{1a}═H.

In schemes 2a and 3a, the RCOCl reagent in the first stage, cinnamoyl chloride, may be replaced by (2E)-3-ethyloxy-2-propenoyl chloride and the subsequent cyclisation effected with trifluoroacetic acid or sulfuric acid instead of aluminium trichloride (E. Baston et al, European J. Med. Chem., 2000 35(10), 931.

A route to intermediate (IV) where Z is N is shown in Scheme 2b. Acrylate (IV) may be prepared by reaction of triflate (V) where R^{1a} is H or Cl with the known stannane (for synthesis of this stannane see Zhang, H. X.; Guibe, F.; Balavoine, G. J. Org. Chem. (1990), 55(6), 1857.) (VI) under typical Stille coupling conditions (for an example see Levin, Jeremy I. Tetrahedron Letters (1993), 34(39), 6211.

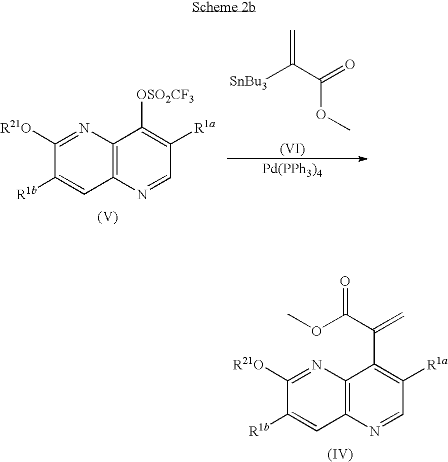

An alternative route to intermediate (IV) , where Z is N, R^{1a} is H, Cl, F, cyano, (C₁₋₆)alkyl is shown in Scheme 3b. L is a leaving group such as triflate or halogen e.g. bromine. For example, reaction of a chlorotriflate (IX) (L=triflate, R^{1a}═Cl) with the sodium salt of dimethylmalonate under basic conditions provides diester (VIII) under conventional conditions (see for an example Fellows, Ingrid M.; Kaelin, David E., Jr.; Martin, Stephen F. J. Am. Chem. Soc., 2000, 122(44), 10781). The palladium catalysed reaction of malonate with bromofluoro derivative (IX) (L=Br, R^{1a}═F) using Pd/Pt—Bu₂ gives (VIII) (for an example see Beare, Neil A.; Hartwig, John F. J. Org. Chem., 2002, 67(2), 541). Also, the copper catalysed reaction of the sodium salt of dimethylmalonate with bromofluoro derivative (IX) (L=Br, R^{1a}═F) or bromochloro derivative (L=Br, R^{1a}═Cl) gives (VIII)(for an example see U.S. Pat. No. 6,156,925).

Diester (VIII) may be decarboxylated to give ester (VII) under standard conditions (for an example see Krapcho, A. Paul; Jahngen, E. G. E., Jr.; Lovey, A. J.; Short, Franklin W. Tetrahedron Lett., 1974, (13), 1091; Krapcho et al., J. Org. Chem. 1987, 52(9), 1880, by heating a mixture of diester with LiCl in DMSO/water at 100° C. Conversion of (VII) to the acrylate (IV) may be effected by reaction with paraformaldehyde under basic conditions (for an example see Serelis, Algirdas K.; Simpson, Gregory W. Tetrahedron Lett. 1997, 38(24), 4277.

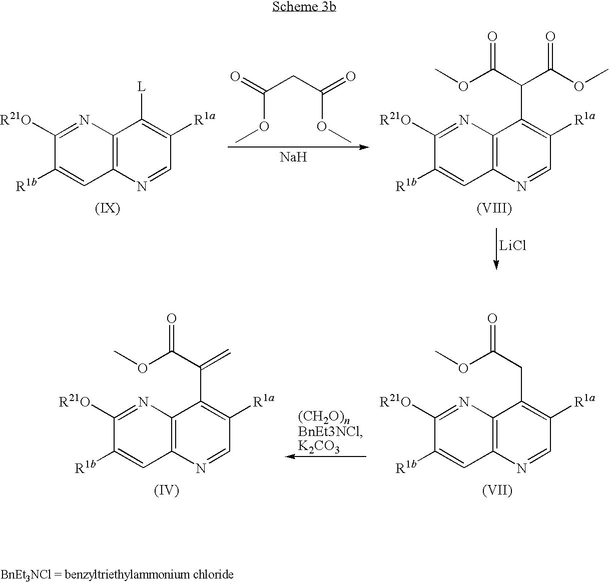

An alternative route to compounds of formula (I) in which Z is CH, A is (ia), n is 1 and R³ is H and U is CH₂ comprises reaction of a compound of formula (IIE):

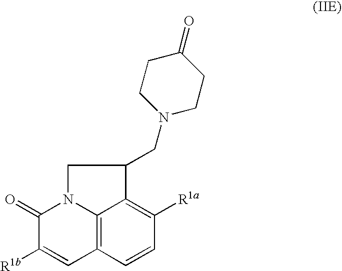

where R^{1a} and R^{1b} are as described in formula (I), with a compound R⁵NH₂, by reductive alkylation.

The compound of formula (IIE) may be prepared by the following Scheme 4:

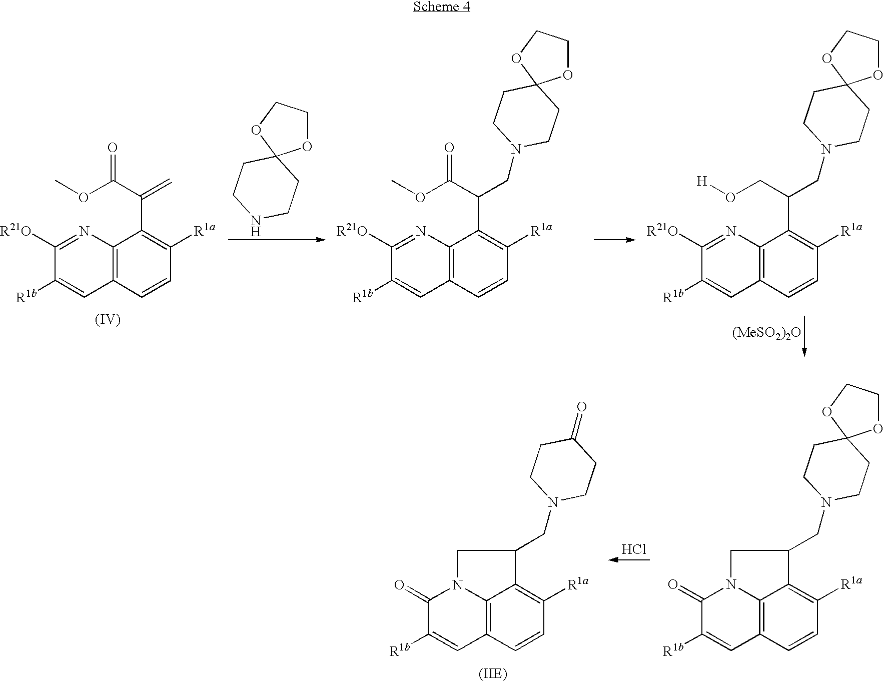

Reaction of (IV) with a suitable protected ketopiperidine such as 1,4-dioxa-8-azaspiro[4.5]decane followed by reduction of the ester and cyclisation with methane sulphonic anhydride gives the tricylic intermediate. Deprotection of the acetal with hydrochloric acid liberates the ketone.

Another alternative route to compounds of formula (IIB) in which Z is CH, R^{1a} is F, R^{1b} is H, R⁹ is H, R²⁰ is H, R^{2′} is Boc, A is (ia), n is 1 and R³ is H (compound 5), comprises Scheme 5A:

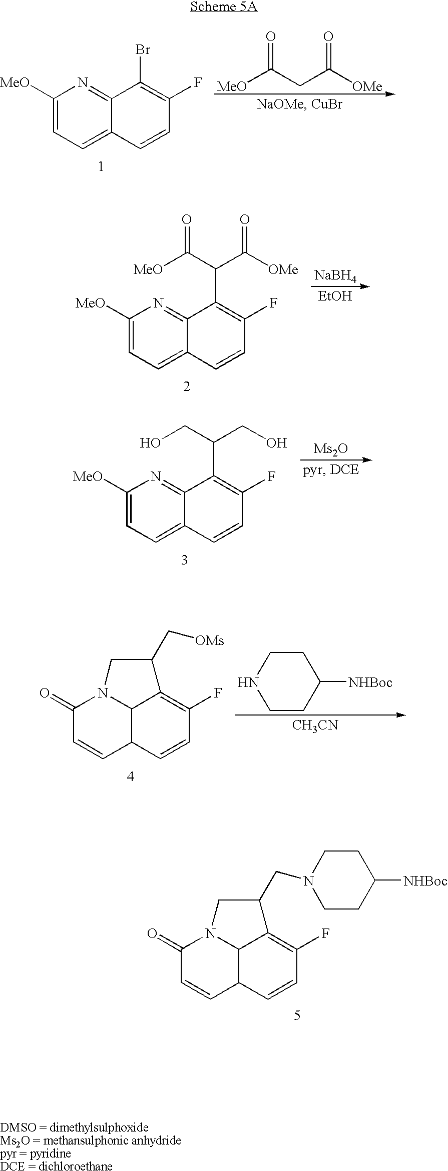

The diol 3 may be subjected to an enzymatic desymmetrization reaction to generate the desired E1 enantiomer of compound 4, by treatment with lipase TL and a vinyl ester (such as vinyl acetate or vinyl pivalate), followed by cyclisation with methanesulphonic anhydride, ester hydrolysis with sodium methoxide in methanol and activation of the resultant alcohol to mesylate 4 by conventional methods. A variant of this process is shown in Scheme 5B:

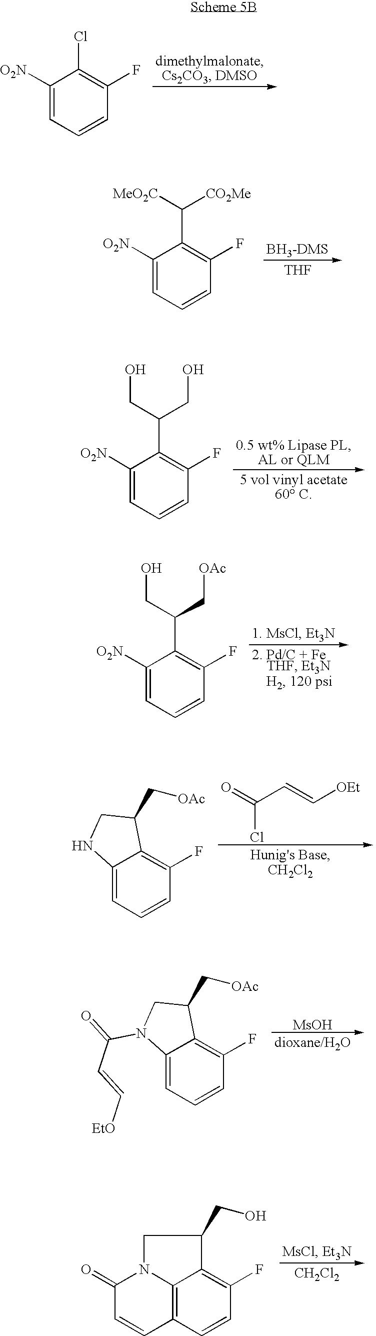

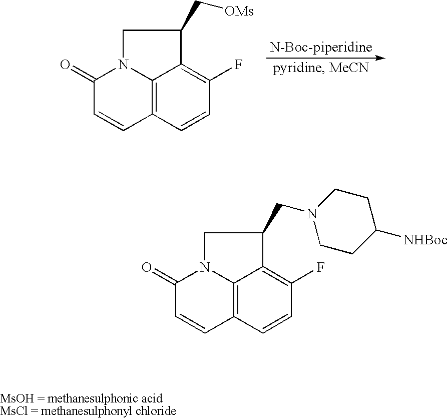

In a further aspect of the invention there is provided a process for preparing compounds of formula (I) and pharmaceutically acceptable salts thereof, in which Z is N, which process comprises reacting a compound of formula (IIF):

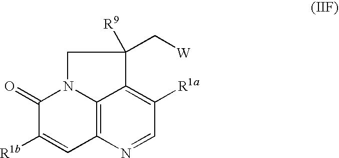

with a compound HA-N(R²⁰)R^{2′} in which W is a leaving group, R²⁰ is R⁵ or a group convertible thereto and R^{2′} is R² or a group convertible thereto, and A, R^{1a}, R^{1b}, R², R⁹, R⁵ are as defined in formula (I), to give a compound of formula (IIG):

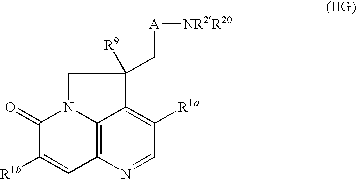

and and thereafter optionally or as necessary converting R²⁰ and R^{2′} to R⁵ and R², interconverting any variable groups, and/or forming a pharmaceutically acceptable salt thereof.

The reaction is carried out under conventional conditions for amine coupling such as reacting together in the presence of a suitable base, such as sodium carbonate or triethylamine, in a suitable solvent such as ethanol or N,N-dimethylformamide at temperatures between ambient and 60° C. Where R⁹ is OH, treatment with base can afford an epoxide which can react with amines to give (IIG). Such reactions may proceed through this epoxide without the need for isolation.

The compound of formula (IIF) may be prepared by the following Scheme 6:

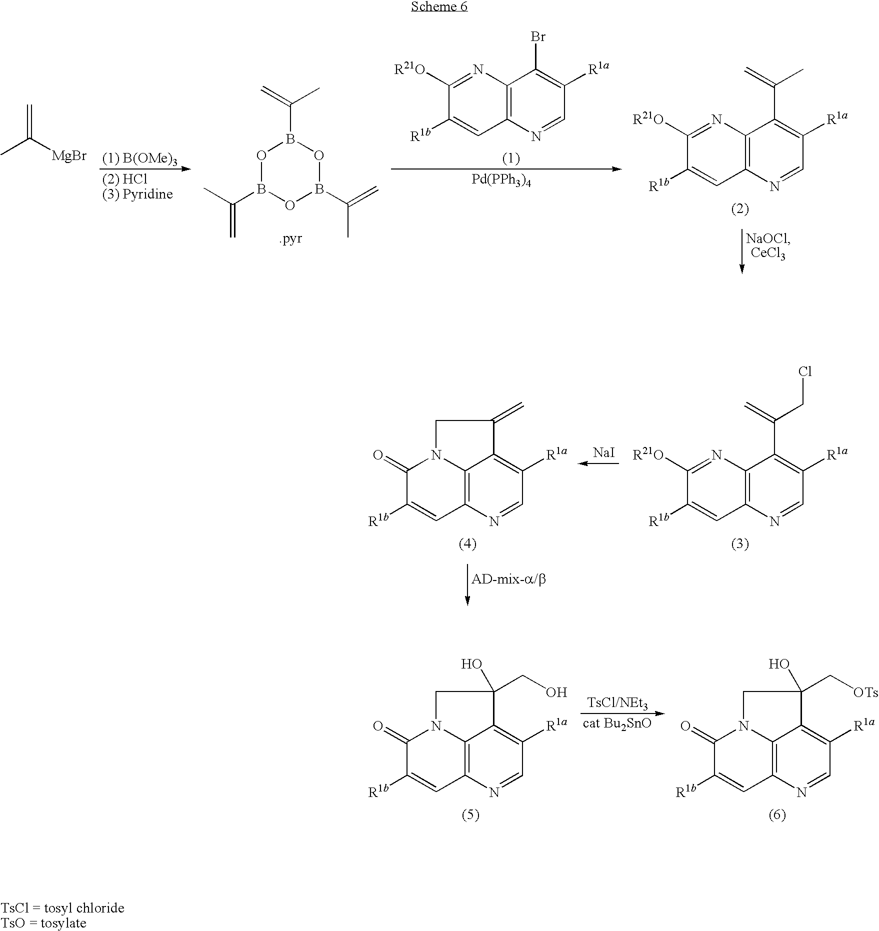

The bromo-naphthyridine (1) is converted to a methylvinyl-analogue (2) under Suzuki conditions. The methyl group is functionalised with NaOCl to give the chloroanalogue (3) which cyclises to give the vinyl tricyclic naphthyridone (4). The vinyl tricyclic naphthyridone (4) is converted to the dihydroxylated analogue (5) using AD-mix-α and/or β, a mixture of potassium osmate, potassium ferricyanide and chiral alkaloid-derived ligand known to dihydroxylate olefins in a chiral manner, see K. B. Sharpless et al, Chem. Rev., 1994, 94, 2483. Alternative chiral ligands may also be used such as hydroquinine anthraquinone-1,4-diyl diether. The primary hydroxyl group is functionalised to the leaving group W of the compound of formula (IIA) conventionally, for example to the tosylate (6) with tosyl chloride/dibutyltin oxide.

An alternative route to a vinyl derivative (4) in which R^{1a} is F and R^{1b} is H is shown in Scheme 7:

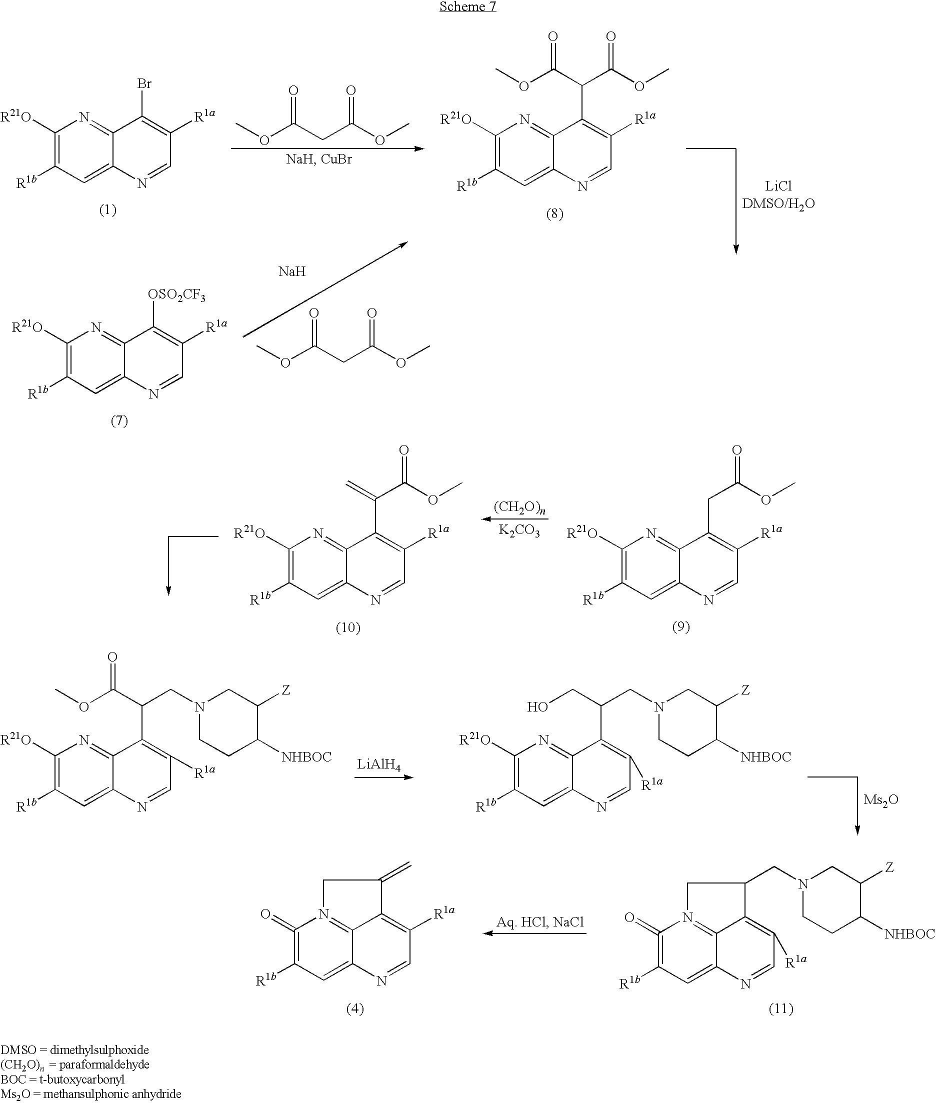

Bromides such as (1) can be converted to diester (8) by copper-catalysed reaction with the sodium salt of dimethyl malonate, while triflates such as (7) can be converted directly to (8) by reaction with the sodium salt of dimethyl malonate. The diesters can be converted to monoesters (9) using the conditions of Krapcho et al, J. Org. Chem., 1987, 52(9), 1880, by heating a mixture of diester with LiCl in DMSO/water at 100° C. for 24 h. Condensation with paraformaldehyde gives the propenoate (10) then the sequence of Michael addition with an aminopiperidine (Z is H or OH), reduction of ester to alcohol, and cyclisation gives the tricyclic derivatives (11). Acid-catalysed retro-Michael reaction then gives the key olefins (4), which can then be further transformed according to the methods described in Scheme 6.

R⁹ hydroxy may be converted to fluoro at any point in the synthesis, such as on intermediate (5), by treatment with a fluorinating agent such as (diethylamino)sulphur trifluoride.

Conveniently the resolution of enantiomers at the attachment position of R⁹ is carried out on the compound of formula (I), (IIB) or (IIG), by any conventional method such as preparative high performance liquid chromatography.

Interconversions of R^{1d}, R^{1b}, R², A and R⁵ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

Interconversion of R^{1a} and R^{1b} groups may be carried out conventionally, on compounds of formula (I) or (IIB). For example R^{1a} or R^{1b} methoxy is convertible to R^{1a} or R^{1b} hydroxy by treatment with lithium and diphenylphosphine (general method described in Ireland et al, J. Amer. Chem. Soc., 1973, 7829) or HBr. Alkylation of the hydroxy group with a suitable alkyl derivative bearing a leaving group such as halide, yields R^{1a} or R^{1b} substituted alkoxy. R^{1a} halogen is convertible to other R^{1a} by conventional means, for example to hydroxy, alkylthiol (via thiol) and amino using metal catalysed coupling reactions, for example using copper as reviewed in Synlett (2003), 15, 2428-2439 and Angewandte Chemie, International Edition, 2003, 42(44), 5400-5449. R^{1b} halo such as bromo may be introduced by the method of M. A. Alonso et al, Tetrahedron 2003, 59(16), 2821. R^{1a} or R^{1b} halo such as bromo may be converted to cyano by treatment with copper (I) cyanide in N,N-dimethylformamide. R^{1a} or R^{1b} carboxy may be obtained by conventional hydrolysis of R^{1a} or R^{1b} cyano, and the carboxy converted to hydroxymethyl by conventional reduction.

Compounds of formula HA-N(R²⁰)R^{2′} and (V) are known compounds or may be prepared analogously to known compounds, see for example WO0224684, WO2004/035569, WO2004/089947, WO02/08224, WO02/50061, WO02/56882, WO02/96907, WO2003087098, WO2003010138, WO2003064421, WO2003064431, WO2004002992, WO2004002490, WO2004014361, WO2004041210,WO2004096982, WO2002050036, WO2004058144, WO2004087145, WO2003082835, WO2002026723, WO06002047, WO06014580, WO06134378, WO06137485, WO07016610, WO07081597, WO07071936, WO07115947, WO07118130, WO07122258, WO08006648, WO08003690 and WO08009700.

As shown in Scheme 8, the hydroxy-aminomethylpyrrolidines of formula (XIII) (HA-NH(R²⁰), A is (ii), X is CR⁴R⁸, W¹ is a bond, W² and W³ are both CH₂, R⁴ and R⁷ are H and R⁸ is OH) can be prepared from doubly protected chiral intermediate

(XVI), separated by preparative HPLC. The benzyloxycarbonyl protecting group is removed by hydrogenation to give (XV) and the amino function converted to a trifluoroacetamide (XIV). The t-butoxycarbonyl (Boc) protecting group is removed with HCl to give the pyrrolidine hydrochloride salt (III).

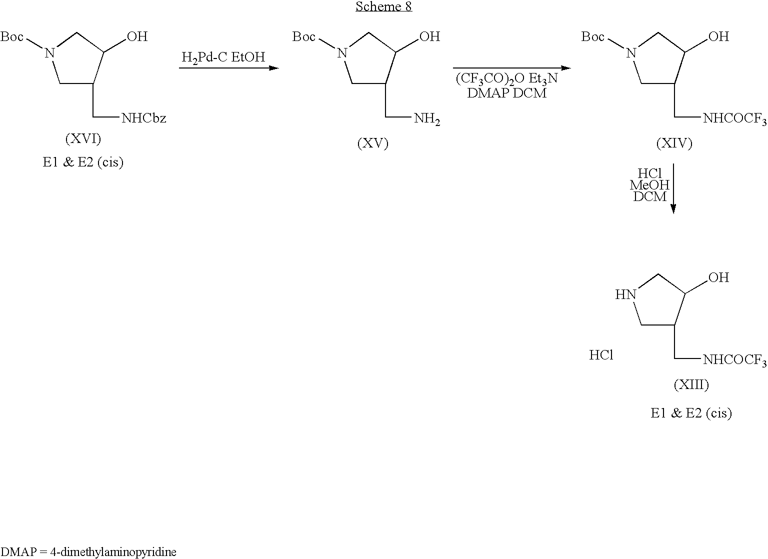

The intermediate (XVI) may be prepared by the general method of Scheme 9:

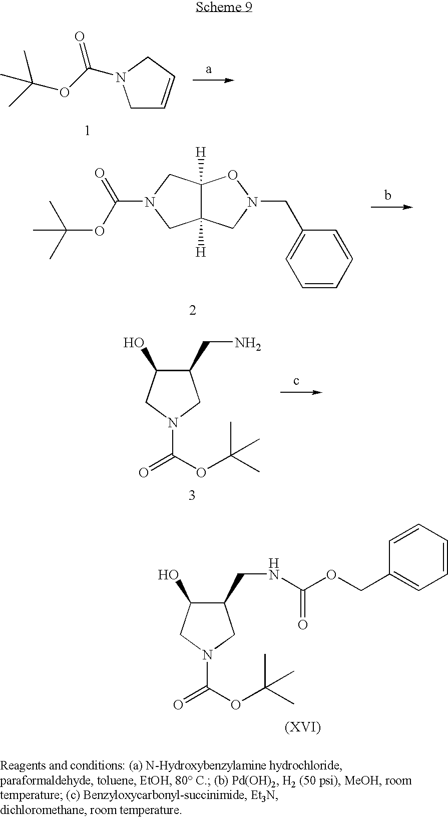

In Scheme 10 the aminomethylpyrrolidine of formula (XVII) (HA-NH(R²⁰), A is (ii), X is CR⁴R⁸, W¹ is a bond, W² and W³ are both CH₂, R⁴, R⁷ and R⁸ are all H) can be prepared from commercially available Boc-protected aminomethylpyrrolidine, and converted to the trifluoroacetamide.

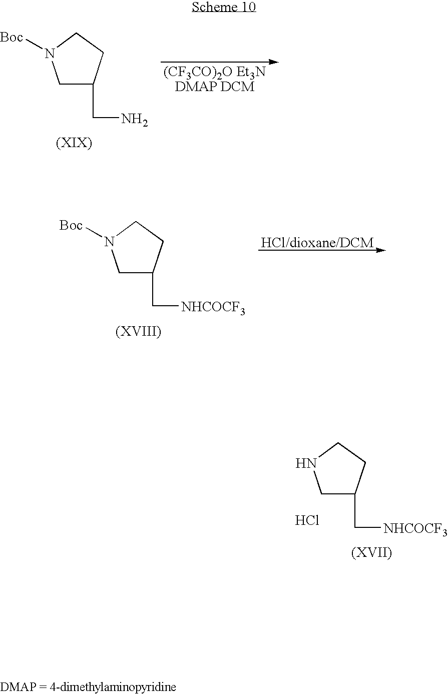

The aminomethylmorpholine intermediate of formula (XXI) (HA-NH(R²⁰), A is (ii), X is O, W¹, W² and W³ are each CH₂) may be prepared from a chiral dichlorobenzyl intermediate (XXIII) (WO2003082835) (Scheme 9) by first protecting the amino function with a Boc-protecting group (XXII), removing the dichlorobenzyl group by hydrogenation to give (XXI), protecting the morpholine N-atom with a benzyloxycarbonyl group (to allow purification by chromatography) (XX), and hydrogenation to afford the required morpholine derivative (XXI).

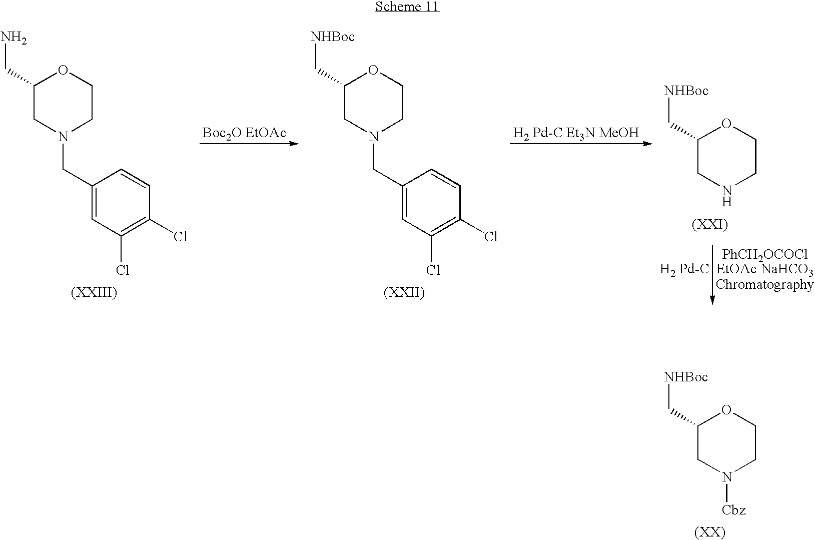

R⁵-halides and R⁵-W derivatives, acyl derivatives or aldehydes are commercially available or are prepared conventionally. The aldehydes may be prepared by partial reduction of the corresponding ester with lithium aluminium hydride or di-isobutylaluminium hydride or more preferably by reduction to the alcohol, with lithium aluminium hydride or sodium borohydride (see Reductions by the Alumino- and Borohydrides in Organic Synthesis, 2nd ed., Wiley, N.Y., 1997; JOC, 3197, 1984; Org. Synth. Coll., 102, 1990; 136, 1998; JOC, 4260, 1990; TL, 995, 1988; JOC, 1721, 1999; Liebigs Ann./Recl., 2385, 1997; JOC, 5486, 1987) , followed by oxidation to the aldehyde with manganese (II) dioxide. The aldehydes may also be prepared from carboxylic acids in two stages by conversion to a mixed anhydride for example by reaction with isobutyl chloroformate followed by reduction with sodium borohydride (R. J. Alabaster et al., Synthesis, 598, 1989) to give the hydroxymethyl substituted heteroaromatic or aromatic and then oxidation with a standard oxidising agent such as pyridinium dichromate or manganese (II) dioxide. Acyl derivatives may be prepared by activation of the corresponding ester. R⁵-halides such as bromides may be prepared from the alcohol R⁵OH by reaction with phosphorus tribromide in dichloromethane/triethylamine. Where X^{2a} is CO and X^{3a} is NR^{13a} the R⁵-halide may be prepared by coupling an X^{4a}—NH₂ amine and bromoacetyl bromide. R⁵—W derivatives such as methanesulphonyl derivatives may be prepared from the alcohol R⁵OH by reaction with methane sulphonyl chloride. The leaving group W may be converted to another leaving group W, e.g. a halogen group, by conventional methods.

The amines R²′R^{4′}NH are available commercially or prepared conventionally. For example amines may be prepared from a bromo derivative by reaction with sodium azide in dimethylformamide (DMF), followed by hydrogenation of the azidomethyl derivative over palladium-carbon. An alternative method is to use potassium phthalimide/DMF to give the phthalimidomethyl derivative, followed by reaction with hydrazine in DCM to liberate the primary amine.

Amines where X^{2a} is CO and X^{3a} is NR^{13a} may be prepared by reacting an N-protected glycine derivative HO₂C—X^{1a}—NH₂ with X^{4a}—NH₂ by conventional coupling using eg 1-ethyl-3-[3-dimethylaminopropyl]carbodiimide.

Further details for the preparation of compounds of formula (I) are found in the examples.

The antibacterial compounds according to the invention may be formulated for administration in any convenient way for use in human or veterinary medicine, by analogy with other antibacterials/antitubercular compounds.

The pharmaceutical compositions of the invention may be formulated for administration by any route and include those in a form adapted for oral, topical or parenteral use and may be used for the treatment of bacterial infection including tuberculosis in mammals including humans.

The compositions may be in the form of tablets, capsules, powders, granules, lozenges, suppositories, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propylp-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-1000 mg of the active ingredient. The dosage as employed for adult human treatment will preferably range from 100 to 3000 mg per day, for instance 1500 mg per day depending on the route and frequency of administration. Such a dosage corresponds to about 1.5 to about 50 mg/kg per day. Suitably the dosage is from 5 to 30 mg/kg per day.

The compound of formula (I) may be the sole therapeutic agent in the compositions of the invention or a combination with other antibacterials including antitubercular compounds. If the other antibacterial is a β-lactam then a β-lactamase inhibitor may also be employed.

Compounds of formula (I) may be used in the treatment of bacterial infections caused by a wide range of organisms including both Gram-negative and Gram-positive organisms, such as upper and/or lower respiratory tract infections, skin and soft tissue infections and/or urinary tract infections. Compounds of formula (I) may be also used in the treatment of tuberculosis caused by Mycobacterium tuberculosis. Some compounds of formula (I) may be active against more than one organism. This may be determined by test methods described herein.

The following examples illustrate the preparation of certain compounds of formula (I) and the activity of certain compounds of formula (I) against various bacterial organisms including Mycobacterium tuberculosis.

### Examples and Experimental

### General

Abbreviations in the examples:
rt=room temperature
MS=mass spectrum
ES=Electrospray mass spectroscopy
LCMS or LC-MS=Liquid chromatography mass spectroscopy
HPLC=High Performance Liquid Chromatography (Rt refers to retention time)
MDAP or Mass directed autoprep=mass directed preparative HPLC
   Certain reagents are also abbreviated herein. DMF refers to N,N-dimethylformamide, TFA refers to trifluoroacetic acid, THF refers to tetrahydrofuran, Pd/C refers to palladium on carbon catalyst, DCM refers to dichloromethane, Boc refers to tert-Butoxycarbonyl, MeOH refers to methanol, NMP refers to N-methyl-2-pyrrolidone, AcOH refers to acetic acid, DMSO refers to dimethylsulfoxide, Pol-BH₄ refers to Borohydride on Amberlite IRA-400, Macroporous, 20-50 mesh, ((Polystyrenemethyl)trimethylammonium borohydride), DIBAL-H refers to diisobutylaluminium hydride.

Proton nuclear magnetic resonance (¹H NMR) spectra were recorded at 400 or 250 MHz, and chemical shifts are reported in parts per million (6) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad, J indicates the NMR coupling constant measured in Hertz. CDCl₃ is deuteriochloroform, DMSO-d₆ is hexadeuteriodimethylsulfoxide, and CD₃OD is tetradeuteriomethanol. Mass spectra were obtained using electrospray (ES) ionization techniques. All temperatures are reported in degrees Celsius.

MP-carbonate refers to macroporous triethylammonium methylpolystyrene carbonate (Argonaut Technologies). Chiralpak AD and AD-H columns comprise of silica for preparative columns (Sum particle size AD-H and 10 um particle size AD 21×250 mm; 20 um particle size AD, 101.1×250 mm) coated with Amylose tris (3,5-dimethylphenylcarbamate) (Chiral Technologies USA). Chiralpak AS-H column comprise of amylose tris[(S)-alpha- methylbenzylcarbamate) coated onto Sum silica. Chiralpak IA column comprise of amylose tris (3,5- dimethylphenylcarbamate) immobilized onto Sum silica. Luna™ C18 semi-prep reverse phase columns comprise of silca particles coated at high density with C18 alkyl chains and have good acid stability within a wide pH range (pH1.5 to pH10). The SCX (Strong Cation eXchange) column has benzene sulphonic acid covalently attached to a silica support and as such strongly retains high pKa (ie basic) organic molecules such as amines, which can be subsequently liberated with excess ammonia in an appropriate solvent. Measured retention times are dependent on the precise conditions of the chromatographic procedures. Where quoted below in the Examples they are indicative of the order of elution.

Reactions involving metal hydrides including lithium hydride, lithium aluminium hydride, di-isobutylaluminium hydride, sodium hydride, sodium borohydride and sodium triacetoxyborohydride are carried out under argon or other inert gas.

As will be understood by the skilled chemist, references to preparations carried out in a similar manner to, or by the general method of, other preparations, may encompass variations in routine parameters such as time, temperature, workup conditions, minor changes in reagent amounts etc.

### EXAMPLE 1

### (4S)-3-Fluoro-4-hydroxy-4-[(4-{[(2E)-3-(2-pyridinyl)-2-propen-1-yl]amino}-1-piperidinyl)methyl]-4,5-dihydro-7H-pyrrolo[3,2,1-de]-1,5-naphthyridin-7-one dihydrochloride

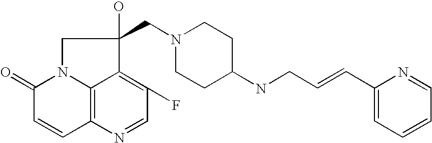

### (a) Pyridine-tris(1-methylethenyl)boroxin (1:1)

A suspension of magnesium turnings (20.83 g, 868 mmol) in THF (1.2 litres) under argon at room temperature was treated with 2-bromopropene (20 ml) and the reaction initiated with a little heat. Upon initiation, external heating was stopped and the reaction mixture maintained at around 50-60° C. by the addition of more 2-bromopropene in three portions (53 ml) over a period of around 1.5 h. The mixture, containing isopropenylmagnesium bromide, was allowed to cool to ambient temperature. The remainder of the procedure followed that as described for the corresponding pyridine-tris(triethenyl)boroxin (1:1) (J. Org. Chem. 2002, 67, 4968). The solution of isopropenylmagnesium bromide (approximately 826 mmol in 1.2 litres of THF) and an additional batch of isopropenylmagnesium bromide 250 mmol in 500 ml of THF (commercial material) was added to a solution of trimethyl borate (217 ml, 1937 mmol) in 1 litre THF cooled to −78° C. under argon. After the addition was complete (approximately 1 hour) the mixture was maintained at −78° C. for 1 hour before the addition of 1M HCl (550 ml) over 5 min. The mixture was allowed to warm to ambient temperature, then brine (500 ml) and diethyl ether (500 ml) were added. The aqueous phase was further extracted with diethyl ether (3×500 ml) and the combined organic extracts were washed with water (500 ml), brine (500 ml), dried (sodium sulphate) and evaporated to a volume of approximately ⅛. Pyridine (220 ml) was added and the mixture stirred for a period of 4 hours. Evaporation afforded an oil (86.4 g, 83%).

δH (CDCl₃, 400 MHz) 1.80 (9H, s), 5.45 (3H, s), 5.65 (3H, s), 7.62 (2H, t), 8.02 (1H, m), 8.85 (2H, d).

Catalytic iodine may be used to initiate the reaction of 2-bromopropene with magnesium. Excess boric acid and derivatives thereof may be removed from the reaction mixture by filtration through Celite® before addition of pyridine.

### (b) 7-Fluoro-8-(1-methylethenyl)-2-(methyloxy)-1,5-naphthyridine

### Method A

A solution of 8-bromo-7-fluoro-2-(methoxy)-1,5-naphthyridine (8.53 g, 33.2 mmol) (for a synthesis, see WO2004058144, Example 53 (g)) and tetrakis(triphenylphosphine)palladium(0) (1.92 g, 1.7 mmol) in degassed dimethoxyethane (300 ml) was stirred under argon for 30 minutes. Potassium carbonate (4.58 g, 33.2 mmol), water (90 ml) and pyridine-tris(1-methylethenyl)boroxin (1:1) (3.8 g, 13.3 mmol) were added and the mixture was heated to reflux for 10 hours. The mixture was allowed to cool and treated with water (500 ml) and diethyl ether (500 ml). The phases were separated and the aqueous phase further extracted with diethyl ether (3×500 ml). The combined organic extracts were dried over magnesium sulphate and evaporated. The residue was chromatographed on silica eluting with a gradient of 0-50% ethyl acetate in hexane affording a yellow oil (6.9 g, 95%).

MS (+ve ion electrospray) m/z 219 (MH+).

### Method B

To a flask were charged THF (50 ml) and magnesium turnings (1.9 g, 2 eq) excluding air. The mixture was stirred at room temperature for 30-60 minutes. A first 10-20% portion of 2-bromopropene (6.9 ml, 9.4 g, 1.3 eq in all) in THF (20 mL) was added. A catalytic amount of iodine and heating to 40° C. were employed to initiate Grignard reaction then allowed to naturally cool. A temperature rise was observed when Grignard reaction initiated. The remaining 2-bromopropene was added in while maintaining temperature below 62° C. The resulting mixture after addition was at ˜50° C. and was heated at ˜47° C. to ensure the reaction was complete.

To a separate flask was charged THF (20 ml). Solid zinc chloride (20 g, 2.5 eq) was added in one portion and a temperature rise was observed. The mixture was cooled down to ˜10° C.

The above freshly prepared Grignard reagent (in THF) was transferred into the ZnCl₂ mixture slowly at a rate maintaining temperature below 25° C. The resulting mixture was warmed up to room temperature, a further 20 ml THF was added and stirred at room temperature for ˜2 hours.

To the above reaction mixture were added tetrakis(triphenylphosphine)palladium(0) [Pd(PPh3)4, 1.3 g, 2% mol] as a solid in one portion and 8-bromo-7-fluoro-2-(methoxy)-1,5-naphthyridine (15.0 g, 1 eq) as a solid in one portion. No heat was observed. The reaction mixture was heated at reflux until reaction complete monitoring with HPLC.

The reaction was cooled down to ˜10° C. and quenched with 100 ml of 10% citric acid aqueous solution (exothermic). Ethyl acetate (100 ml) was added for extraction. The organic layer was washed with 100 ml water and the aqueous layer was extracted with 100 ml ethyl acetate. The combined organic solution was concentrated to dryness to afford a dark oil and further purified by a silica column with heptane/ethyl acetate (1/0, 3/1, 2/1) as the eluent (11.8 g, ˜93% yield and ˜97% purity per area ratio by HPLC).

Zinc bromide may be substituted for zinc chloride, and the concentration of the palladium catalyst may be reduced to 0.5%. The washing step may be performed with 4% citric acid aqueous solution instead of water.

### (c) 8-[1-(Chloromethyl)ethenyl]-7-fluoro-2-(methyloxy)-1,5-naphthyridine

### Method A

A solution of 7-fluoro-8-(1-methylethenyl)-2-(methyloxy)-1,5-naphthyridine (7.3 g, 33.5 mmol) in tent-butanol (500 ml) under argon was treated with cerium(III) chloride heptahydrate (12.5 g, 33.5 mmol) followed by a dropwise solution of sodium hypochlorite (12% w/v, 21.1 ml, 33.5 mmol). A yellow suspension was formed. After 15 minutes more sodium hypochlorite (12% w/v, 21.1 ml, 33.5 mmol) was added. After 10 minutes stirring, saturated aqueous sodium sulphite solution (200 ml) was added. After 5 minutes stirring water (500 ml) was added and the mixture extracted with ether (3×500 ml). The combined organic extracts were dried over magnesium sulphate and evaporated. The residue was chromatographed on silica eluting with a gradient of 0-50% ethyl acetate in hexane affording product (4.9 g, 58%).

The organic extracts may be dried by washing with brine and ethyl acetate instead of drying over magnesium sulphate.

### Method B

A solution of 7-fluoro-8-(1-methylethenyl)-2-(methyloxy)-1,5-naphthyridine (31.86 g, 146 mmol) in tert-butanol (2 L) was treated with cerium(III) chloride heptahydrate (54.4 g, 146 mmol), stirred for 15 minutes then treated with a solution of sodium hypochlorite (12% w/v, 139 ml, 221 mmol) added over 15 minutes. After 15 minutes stirring saturated aqueous sodium sulphite solution (800 ml) was added. After 30 minutes stirring more water was added and the mixture extracted with diethyl ether (1×500 ml, 2×2 L). The combined organic extracts were dried and evaporated. The residue was chromatographed on silica eluting a gradient of 0-100% dichloromethane in hexane affording product (17.5 g, 48%).

MS (+ve ion electrospray) m/z 253 (MH+).

### (d) 3-Fluoro-4-methylidene-4,5-dihydro-7H-pyrrolo[3,2,1-de]-1,5-naphthyridin-7-one

A mixture of 8-[1-(chloromethyl)ethenyl]-7-fluoro-2-(methyloxy)-1,5-naphthyridine (4.9 g, 19.5 mmol) and sodium iodide (29 g, 195 mmol) in acetone (100 ml) was heated to reflux for 18 hours. The mixture was evaporated and the residue partitioned between water (200 ml) and dichloromethane (200 ml). The aqueous phase was further extracted with dichloromethane (2×200 ml) and the combined dichloromethane extracts washed with water (2×200 ml) dried over magnesium sulphate and evaporated. The resulting solid was triturated with ethyl acetate (50 ml) and filtered and washed with ethyl acetate (20 ml) affording a brown solid. This material was then dissolved in ethyl acetate and filtered through a plug of silica. Evaporation afforded a solid (1.4 g, 36%).

MS (+ve ion electrospray) m/z 203 (MH+).

If necessary the product may be taken up in n-hexane and precipitated out and the solid purified by stirring with cold acetone.

### (e) 3-Fluoro-4-hydroxy-4-(hydroxymethyl)-4,5-dihydro-7H-pyrrolo[3,2,1-de]-1,5-naphthyridin-7-one

### Method A

A mixture of 3-fluoro-4-methylidene-4,5-dihydro-7H-pyrrolo[3,2,1-de]-1,5-naphthyridin-7-one (37.1 g, 183 mmol), tert-butanol (1.5 litres), water (1.5 litres) and AD mix alpha (Aldrich) (246 g) was stirred vigorously for 16 hours at room temperature. Sodium sulphite (300 g) was added and the mixture was stirred for 30 minutes. The phases were separated and the aqueous phase was extracted three times with 10% methanol in ethyl acetate (total volume 1 litre). The combined organic extracts were dried and evaporated affording a solid (45 g, 100%).

MS (+ve ion electrospray) m/z 237 (MH+).

### Method B

To a 1 L 3-neck flask was added potassium hexacyanoferrate (III) (24.50 g), potassium carbonate (granular, 10.25 g), potassium osmate (VI) dihydrate (25 mg), and water (125 mL). This mixture was stirred at room temperature for 10 min, to which was added a predissolved solution of hydroquinine anthraquinone-1,4-diyl diether ((DHQ)2AQN) (200 mg) in 2-butanol (125 mL). The resulting mixture was stirred for 15 min, to which was added 3-fluoro-4-methylidene-4,5-dihydro-7H-pyrrolo[3,2,1-de]-1,5-naphthyridin-7-one (4.60 g). The resulting mixture was stirred at ambient temperature for 18 h. To the mixture was added Na₂SO₃ (37.25 g), and stirred for 30 min at room temperature. To this mixture was added water (100 mL) and methanol (10 mL), extracted with 3×250 mL of ethyl acetate. The combined ethyl acetate fractions were washed with brine, dried over Na₂SO₄, filtered, and concentrated in vacuo to give title compound as a gum. Chiral HPLC indicated the ratio of 4R/4S to be 13.3/86.7.

t-Butanol may be used in place of 2-butanol in the above reaction with (DHQ)2AQN and also for the extraction step in place of ethyl acetate. If necessary the product may be dissolved in warm toluene and cooled to precipitate the product as a solid.

### (f) 1,1-Dimethylethyl{1-[(3-fluoro-4-hydroxy-7-oxo-4,5-dihydro-7H-pyrrolo[3,2,1-de]-1,5-naphthyridin-4-yl)methyl]-4-piperidinyl}carbamate

### Method A

A solution of 3-fluoro-4-hydroxy-4-(hydroxymethyl)-4,5-dihydro-7H-pyrrolo[3,2,1-de]-1,5-naphthyridin-7-one (25.09 g, 106.3 mmol) in dichloromethane (1 litre), THF (1 litre) and N,N-dimethylformamide (100 ml) was treated with triethylamine (22.7 ml, 159 mmol), para-toluenesulphonyl chloride (20.2 g, 106.3 mmol) and dibutyltin oxide (1.32 g, 5.23 mmol). After 16 hours stirring at room temperature, water (400 ml) was added, then the organic phase was separated, washed with aqueous sodium bicarbonate and brine, dried and evaporated to give crude toluenesulphonate (approximately 3:1 4S:4R) containing some corresponding epoxide and DMF (43.8 g). This material was dissolved in ethanol (1.1 litres) and treated with sodium carbonate (33.4 g) and 1,1-dimethylethyl 4-piperidinylcarbamate (28.3 g, 142 mmol). The mixture was stirred over the weekend then evaporated almost to dryness. The residue was partitioned between water and 5% methanol in dichloromethane. The phases were separated and the aqueous phase was further extracted with two portions of 5% methanol in dichloromethane. The combined organic extracts were dried and evaporated. The residue was chromatographed on silica (2 kg) eluting with 2-5% methanol/dichloromethane affording the product (39.94 g, 90%).

MS (+ve ion electrospray) m/z 419 (MH+).

### Method B

3-Fluoro-4-hydroxy-4-(hydroxymethyl)-4,5-dihydro-7H-pyrrolo[3,2,1-de]-1,5-naphthyridin-7-one (3.65 g total, 1.32 g with enantiomeric ratio 4R/4S: 15.7/84.3 and 2.33 g with enantiomeric ratio 4R/4S: 17/83), toluenesulfonyl chloride (2.94 g), dibutyltin (IV) oxide (192 mg), tetrahydrofuran (anhydrous, 146 mL), dichloromethane (anhydrous, 146 mL), triethylamine (3.31 mL) and N,N-dimethylformamide (anhydrous, 14.6 mL) was stirred at room temperature as a suspension for 18 h. The mixture was washed with water, saturated aq. NaHCO₃ and brine, dried over Na₂SO₄, filtered and concentrated in vacuo to give the corresponding toluenesulfonate.

A mixture of the above toluenesulfonate, 1,1-dimethylethyl-4-piperidinylcarbamate (4.13 g), Na₂CO₃ (4.87 g) in ethanol (160 mL) was stirred as a suspension at room temperature for 2.5 days. This mixture was combined with another batch prepared analogously from 1.50 g of 3-fluoro-4-hydroxy-4-(hydroxymethyl)-4,5-dihydro-7H-pyrrolo[3,2,1-de]-1,5-naphthyridin-7-one with enantiomeric ratio 4R/4S: 17/83. The resulting mixture was filtered and the Na₂CO₃ cake was washed with ethanol. The filtrate was concentrated in vacuo and taken up in 300 mL of 5% methanol in dichloromethane. The resulting solution was washed with saturated aq. NaHCO₃ (30 mL), dried over Na₂SO₄, filtered and concentrated in vacuo to give a gum. Trituration in dichloromethane gave 1.27 g of the title compound (14% yield, enantiomeric ratio 4R/4S: 15/85). The filtrate was purified by silica gel column chromatography, resulting in 2.00 g of the title compound (22% yield, enantiomeric ratio 4R/4S: 52/48). The Na₂CO₃ cake mentioned above was taken up in water, extracted with 5% methanol in dichloromethane, providing 3.85 g of title compound (42% yield, enantiomeric ratio: 0/100).

2.0 g of title compound (enantiomeric ratio 4R/4S: 15/85) was dissolved in 45 mL of hot ethanol. The solution was aged at room temperature for 1.5 days, chilled at 0° C. for 5 h, −20° C. for 1 day and filtered to give 1.12 g of material that has a measured enantiomeric ratio 4R/4S of 0/100.

1.050 g of title compound with an enantiomeric ratio 4R/4S of 15/85 and 400 mg with an enantiomeric ratio 4R/4S of 52/48 (the measured enantiomeric ratio 4R/4S of this resulting mixture was 24/76) was dissolved in 40 mL of hot ethanol. The solution was cooled gradually to room temperature, aged at ambient temperature for 2.5 days, chilled at 0° C. for 15 min, filtered, and washed with 3×1.5 mL of cold ethanol and dried, resulting in 580 mg of title compound with a measured enantiomeric ratio 4R/4S of 0/100. The filtrate had a measured enantiomeric ratio 4R/4S of 48/52.

Both the reaction with p-toluensulphonyl chloride and the reaction with 1,1-dimethylethyl 4-piperidinylcarbamate may be carried out in dichloromethane. Potassium carbonate may be used in place of sodium carbonate in the reaction with 1,1-dimethylethyl 4-piperidinylcarbamate. If necessary triethylamine may be added in this reaction to promote completion.

A purification protocol for preparing title compound of high enantiomeric purity (4S) from crude product is as follows:
1. Add methanol followed by water to reaction mass and stir for 5-10 min.
2. Separate the organic layer.
3. Extract aqueous layer with 10% methanol in DCM.
4. Combine organic layers and wash with brine solution.
5. Remove solvent under reduced pressure at 25-35° C. to less than 1/10^{th} of its volume.
6. Add 10% ethyl acetate in n-hexane to the residue.
7. Stir at room temperature for 10-12 h.
8. Filter the solid and dry the solid at 50-60° C. under vacuum for 6-8 h.
9. Charge stage 8 and acetonitrile.
10. Stir the reaction mass at 60-70° C. for 1-2 h.
11. Stir the reaction mass at room temperature for 10-12 h.
12. Raise temp to 50-60° C. and stir for 2-3 h or until the solid from a filtered sample shows undesired isomer: <1.0% by analytical chiral hplc.
13. Filter the isolated solid at 50-60° C. and wash the solid with acetonitrile.
14. Suck dry the solid for 30-45 min.
15. Dry the material in the oven at 50-60° C. under vacuum.

The resulting (4S)-1,1-Dimethylethyl{1-[(3-fluoro-4-hydroxy-7-oxo-4,5-dihydro-7H-pyrrolo[3,2,1-de]-1,5-naphthyridin-4-yl)methyl]-4-piperidinyl}carbamate may be used in stage (g) below, obviating the need for the chiral chromatography step

### (g) 4-[(4-Amino-1-piperidinyl)methyl]-3-fluoro-4-hydroxy-4,5-dihydro-7H-pyrrolo[3,2,1-de]-1,5-naphthyridin-7-one

A solution of 1,1-dimethylethyl{1-[(3-fluoro-4-hydroxy-7-oxo-4,5-dihydro-7H-pyrrolo[3,2,1-de]-1,5-naphthyridin-4-yl)methyl]-4-piperidinyl}carbamate (47.94 g, 115 mmol) in dichloromethane/methanol (870 ml/610 ml) was cooled in an ice bath and treated with a solution of hydrochloric acid in 1,4-dioxane (4M, 1.5 litres). The resulting mixture was stirred at room temperature for 1.5 hours then partially evaporated. Filtration, washing with a little dichloromethane and drying in vacuo afforded the hydrochloride of the title compound as a solid (51.55 g). Title compound hydrochloride (50.5 g) was dissolved in water (500 ml) and treated with saturated aqueous sodium carbonate solution until pH8 was obtained (ca 100 ml). The mixture was evaporated to dryness and the resulting solid extracted with 15% methanol in chloroform (3×500 ml). The extracts were evaporated separately affording 22 g, 10 g, and 4 g of solid materials which were essentially identical by spectroscopic analysis.

Title compound (96.5 g) was chromatographed on a Chiralpak AD column eluting with acetonitrile:isopropyl alcohol:isopropylamine 80:20:0.1 affording firstly the E1 enantiomer (4S)-4-[(4-amino-1-piperidinyl)methyl]-3-fluoro-4-hydroxy-4,5-dihydro-7H-pyrrolo[3,2,1-de]-1,5-naphthyridin-7-one (66.1 g) then the E2 enantiomer (4R)-4-[(4-amino-1-piperidinyl)methyl]-3-fluoro-4-hydroxy-4,5-dihydro-7H-pyrrolo[3,2,1-de]-1,5-naphthyridin-7-one (24.4 g).

MS (+ve ion electrospray) m/z 319 (MH+).

This reaction may alternatively be performed using c.HCl in dichloromethane as solvent. The product as the hydrochloride salt may be be precipitated from acetone and used directly in stage (h) below after neutralisation using anhydrous sodium acetate.

### (h) Title Compound

A mixture of (4S)-4-[(4-amino-1-piperidinyl)methyl]-3-fluoro-4-hydroxy-4,5-dihydro-7H-pyrrolo[3,2,1-de]-1,5-naphthyridin-7-one (80 mg, 0.25 mmol) and (2E)-3-(2-pyridinyl)-2-propenal (0.20 mmol) in 1% acetic acid in methanol (7 mL) was shaken for 2 minutes then (polystyryl)methyltrimethylammonium cyanoborohydride (200 mg, 4.0 mmol/g, 0.8 mmol) was added. The mixture was shaken for 1 day then filtered and evaporated. The residue was purified by chromatography on silica eluting with 0-15% 2M ammonia/methanol in dichloromethane affording the free base of the title compound as a yellow gum (35 mg).

δH (CDCl₃, 250 MHz) 1.40-2.00 (4H, m), 2.40 (1H, t), 2.40-2.60 (2H, m), 2.85 (1H, d), 2.90-3.15 (2H, m), 3.25 (1H, d), 3.55 (2H, d), 4.35 (1H, d), 4.45 (1H, d), 6.60-6.85 (3H, m), 7.15 (1H, m), 7.28 (1H, m), 7.60 (1H, t) 7.90 (1H, d), 8.38 (1H, s), 8.55 (1H, m).

MS (+ve ion electrospray) m/z 436 (MH+).

The free base of the title compound was dissolved in dichloromethane (2 mL) and treated with 1M HCl in ether (0.5 mL) and then diluted with ether (5 mL). The resultant precipitate was isolated by centrifugation affording the title compound as a white solid (41 mg).

### EXAMPLE 2

### (4S)-3-Fluoro-4-[(4-{[(2E)-3-(2-furanyl)-2-propen-1-yl]amino}-1-piperidinyl)methyl]-4-hydroxy-4,5-dihydro-7H-pyrrolo[3,2,1-de]-1,5-naphthyridin-7-one dihydrochloride

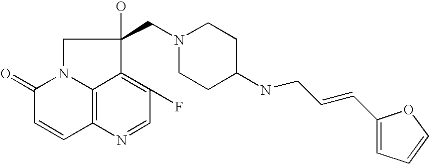

A solution of (4S)-4-[(4-amino-1-piperidinyl)methyl]-3-fluoro-4-hydroxy-4,5-dihydro-7H-pyrrolo[3,2,1-de]-1,5-naphthyridin-7-one (for a synthesis see Example 1(g), enantiomer E1) (80 mg, 0.25 mmol) and (2E)-3-(2-furanyl)-2-propenal (24 mg, 0.2 mmol) in dichloromethane/methanol (1 mL/0.2 mL) was treated with sodium triacetoxyborohydride (127 mg). After 19 hours the reaction mixture was treated with saturated aqueous sodium bicarbonate solution (2 mL). The phases were separated and the aqueous phase extracted with 20% methanol/dichloromethane (1 mL). The combined organic extracts were chromatographed eluting with 0-30% methanol in dichloromethane affording the free base of the title compound as an orange gum (47 mg).

MS (+ve ion electrospray) m/z 436 (MH+).

The free base of the title compound was converted to the title compound by the same procedure as for Example 1 affording the title compound as a light brown solid (48 mg).

δH (d-6 DMSO, 400 MHz) 2.10-2.40 (4H, m), 3.20-3.40 (2H, m), 3.65-4.05 (7H, m), 4.45 (1H, d), 4.85 (1H, d), 6.15 (1H, m), 6.55 (2H, m), 6.75 (1H, m), 6.85 (1H,m), 7.55 (1H, m) 7.70 (1H, bs), 8.10 (1H, m), 8.60 (1H, bs), 9.70 (2H, bs), 10.40 (1H, bs)

### EXAMPLE 3

### 9-Fluoro-1-{[4-({2-methyl-3-[4-(1-methylethyl)phenyl]propyl}amino)-1-piperidinyl]methyl}-1,2-dihydro-4H-pyrrolo[3,2,1-ij]quinolin-4-one monotrifluroacetic acid salt

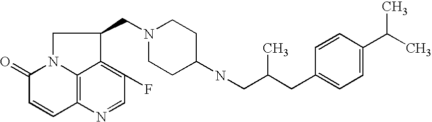

A solution of 1-[(4-amino-1-piperidinyl)methyl]-9-fluoro-1,2-dihydro-4H-pyrrolo[3,2,1-ij]quinolin-4-one (for a synthesis see Preparation 1) (30 mg, 0.1 mmol) in DCM/NMP (0.38 mL/0.12 mL) and 2-methyl-3-[4-(1-methylethyl)phenyl]propanal (53 mg, 0.28 mmol) in DCM/NMP (0.25 mL/0.25 mL) was mixed then was treated with AcOH (0.1 ml) 38% in NMP. The reaction mixture was stirred overnight. Pol-BH₄ (250 mg, 2.5-5 mmol/g) was added, followed by the addition of 1 ml of MeOH. The reaction was stirred overnight. The product was filtered off and solvents were removed under vacuum, then it was re-dissolved in 1:1 DMSO:MeOH (0.5 ml) and purified by MDAP (0% H₂O containing 0.1% TFA-100% CH₃CN containing 0.1% TFA). The title compound was isolated as a TFA salt (15.7 mg). MS (+ve ion electrospray) m/z 476.

### EXAMPLE 4

### (1R)-9-Fluoro-1-[(4-{[(2E)-3-(3-pyridinyl)-2-propen-1-yl]amino}-1-piperidinyl)methyl]-1,2-dihydro-4H-pyrrolo[3,2,1-ij]quinolin-4-one monotrifluroacetic acid salt

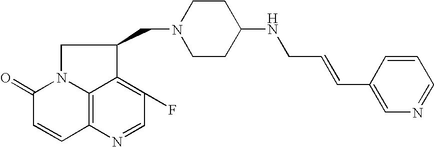

A solution of (1R)-1-[(4-amino-1-piperidinyl)methyl]-9-fluoro-1,2-dihydro-4H-pyrrolo[3,2,1-ij]quinolin-4-one (for a synthesis see Preparation 1, enantiomer E1) (30 mg, 0.1 mmol) in DCM/NMP (0.38 mL/0.12 mL) and (2E)-3-(3-pyridinyl)-2-propenal (37 mg, 0.28 mmol) in DCM/NMP (0.25 mL/0.25 mL) was mixed then was treated with AcOH (0.1 ml) 38% in NMP. The reaction mixture was stirred overnight. Pol-BH₄ (250 mg, 2.5-5 mmol/g) was added, followed by the addition of 1 ml of MeOH. The reaction was stirred overnight. The product was filtered off and solvents were removed under vacuum, then it was re-dissolved in 1:1 DMSO:MeOH (0.5 ml) and purified by MDAP (0% H₂O containing 0.1% TFA-100% CH₃CN containing 0.1% TFA). The title compound was isolated as a TFA salt (28.1 mg). MS (+ve ion electrospray) m/z 419.

### EXAMPLE 5

### (1R)-9-Fluoro-1-[(4-{[(2E)-3-(2-furanyl)-2-propen-1-yl]amino}-1-piperidinyl)methyl]-1,2-dihydro-4H-pyrrolo[3,2,1-ij]quinolin-4-one monotrifluroacetic acid salt

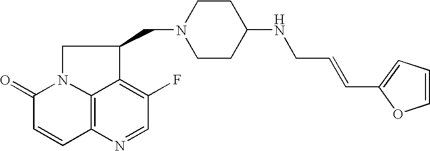

A solution of (1R)-1-[(4-amino-1-piperidinyl)methyl]-9-fluoro-1,2-dihydro-4H-pyrrolo[3,2,1-ij]quinolin-4-one (for a synthesis see Preparation 1, enantiomer E1) (30 mg, 0.1 mmol) in DCM/NMP (0.38 mL/0.12 mL) and (2E)-3-(2-furanyl)-2-propenal (34 mg, 0.28 mmol) in DCM/NMP (0.25 mL/0.25 mL) was mixed then was treated with AcOH (0.1 ml) 38% in NMP. The reaction mixture was stirred overnight. Pol-BH₄ (250 mg, 2.5-5 mmol/g) was added, followed by the addition of 1 ml of MeOH. The reaction was stirred overnight. The product was filtered off and solvents were removed under vacuum, then it was re-dissolved in 1:1 DMSO:MeOH (0.5 ml) and purified by MDAP (0% H₂O containing 0.1% TFA-100% CH₃CN containing 0.1% TFA). The title compound was isolated as a TFA salt (28.9 mg). MS (+ve ion electrospray) m/z 408.

### EXAMPLE 6

### (1R)-1-[(4-{[(2E)-3-(2,5-Difluorophenyl)-2-propen-1-yl]amino}-1-piperidinyl)methyl]-9-fluoro-1,2-dihydro-4H-pyrrolo[3,2,1-ij]quinolin-4-one dihydrochloride

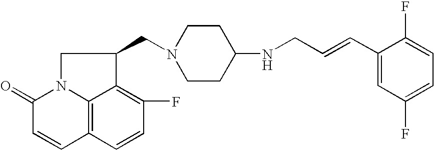

To a 10 mL round-bottomed flask was added (1R)-1-[(4-amino-1-piperidinyl)methyl]-9-fluoro-1,2-dihydro-4H-pyrrolo[3,2,1-ij]quinolin-4-one (for a synthesis see Preparation 1, enantiomer E1) (75 mg, 0.249 mmol) and (2E)-3-(2,5-difluorophenyl)-2-propenal (for a synthesis see Preparation 14) (41.8 mg, 0.249 mmol) in DCM (4 ml) and methanol (0.800 ml) to give a yellow solution. The reaction was allowed to stir 8 h and sodium triacetoxyborohydride (105 mg, 0.498 mmol) was added. The reaction was stirred overnight at rt. The reaction was diluted with DCM (25 mL) and washed with saturated NaHCO₃, followed by saturated NaCl. The organic layer was dried over Na₂SO₄, filtered and concentrated. The crude product was added to a silica gel column and was eluted with 0-15% MeOH in DCM to give the free base of the title compound.

MS (+ve ion electrospray) m/z 454.

1H NMR (400 MHz, CHLOROFORM-d) δ ppm 1.3-1.51 (m, 2H), 1.87-2.00 (m, 2H) 2.11 (td, J=11.3, 2.40 Hz, 1H) 2.26 (td, J=11.3, 2.40 Hz, 1H), 2.51 (dd, J=12.38, 10.86 Hz, 1H), 2.55-2.70 (m, 2H), 2.75-2.85 (m, 1H), 2.88 (dd, J=12.00, 4.42 Hz, 1H) 3.02-3.09 (m, 1H), 3.52 (dd, J=6.32, 1.26 Hz, 2H), 4.03 (m, 1H), 4.45-4.51 (m, 2H), 6.41 (dt, J=15.92, 6.32 Hz, 1H), 6.64 (d, J=9.60 Hz, 1H), 6.70 (s, 1H), 6.85-6.92 (m, 2H), 6.99 (td, J=9.35, 4.55 Hz, 1H), 7.16 (ddd, J=9.16, 5.87, 3.16 Hz, 1H) 7.40 (dd, J=8.59, 4.55 Hz, 1H), 7.68 (d, J=9.60 Hz, 1H).

The free base of the title compound was converted to the title diHCl salt by dissolving product in DCM and treating with 1N HCl in ether and concentrated to give title compound (40 mg, 0.076 mmol, 30.5% yield) as an off white solid.

### EXAMPLE 7

### (1R)-9-Fluoro-1-[(4-{[(2E)-3-(3-fluorophenyl)-2-propen-1-yl]amino}-1-piperidinyl)methyl]-1,2-dihydro-4H-pyrrolo[3,2,1-ij]quinolin-4-one dihydrochloride

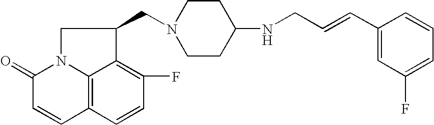

To a 10 mL round-bottomed flask was added (1R)-1-[(4-amino-1-piperidinyl)methyl]-9-fluoro-1,2-dihydro-4H-pyrrolo[3,2,1-ij]quinolin-4-one (for a synthesis see Preparation 1, enantiomer E1) (75 mg, 0.249 mmol) and (2E)-3-(3-fluorophenyl)-2-propenal (for a synthesis see Preparation 15) (37.4 mg, 0.249 mmol) in DCM (4 ml) and methanol (0.800 ml) to give a yellow solution. The reaction was stirred 8 h and sodium triacetoxyborohydride (105 mg, 0.498 mmol) was added. The reaction was stirred overnight at rt. LC MS at t=15 h showed the reaction was incomplete with SM remaining. A second portion of sodium triacetoxyborohydride (105 mg, 0.498 mmol) was added and the reaction was stirred for another 8 h. The reaction was diluted with DCM (25 mL) and washed with saturated NaHCO₃, followed by saturated NaCl. The organic layer was dried with Na₂SO₄, filtered and concentrated. The crude product was added to a silica gel column and was eluted with 0-15% MeOH in DCM to give the free base of the title compound.

MS (+ve ion electrospray) m/z 436.

1H NMR (400 MHz, CDCl₃) δ ppm 1.44-1.60 (m, 2H), 1.87-2.02 (m, 2H), 2.07-2.14 (m, 1H), 2.21-2.28 (m, 1H), 2.50 (dd, J=12.38, 10.86 Hz, 1H) 2.63-2.70 (m, 2H), 2.81-2.87 (m, 2H) 3.04 (d, J=11.62 Hz, 1H), 3.45-3.55 (m, 2H), 4.04 (br. s., 1H), 4.45-4.52 (m, 2H) 6.37 (dt, J=15.73, 6.41 Hz, 1H), 6.53-6.60 (m, 1H) 6.64 (d, J=9.35 Hz, 1H), 6.88 (t, J=8.97 Hz, 1H), 6.93 (dd, J=16.67, 1.77 Hz, 1H), 7.10 (dd, J=10.11, 2.02 Hz, 1H), 7.16 (d, J=7.83 Hz, 1H) 7.25-7.32 (m, 1H), 7.40 (dd, J=8.72, 4.42 Hz, 1H), 7.68 (d, J=9.60 Hz, 1H).

The free base of the title compound was converted to the diHCl salt by dissolving the product in DCM and treating with 1N HCl in ether and concentrated to give title compound (22 mg, 0.043 mmol, 17.39% yield) as an off white solid.

### EXAMPLE 8

### (1R)-9-Fluoro-1-[(4-{[3-(3-fluorophenyl)propyl]amino}-1-piperidinyl)methyl]-1,2-dihydro-4H-pyrrolo[3,2,1-ij]quinolin-4-one dihydrochloride

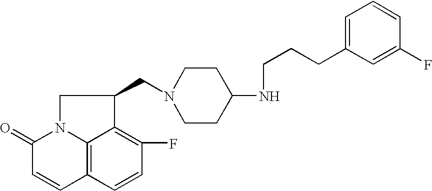

(1R)-1-[(4-Amino-1-piperidinyl)methyl]-9-fluoro-1,2-dihydro-4H-pyrrolo[3,2,1-ij]qin-4-one (for a synthesis see Preparation 1, enantiomer E1) (0.060 g, 0.200 mmol) and 3-(3-fluorophenyl)propanal (for a synthesis see Preparation 16)(0.0305 g, 0.200 mmol) were stirred in DCM (2.5 ml) and methanol (0.5 ml) with sodium sulfate (0.300 g, 2.112 mmol) added to the reaction mixture to facilitate imine formation. After 16.5 hours, sodium triacetoxyborohydride (0.127 g, 0.601 mmol) was added and the mixture stirred for 5.5 hours. Then added an additional (0.069 g, 0.326 mmol) and DCM (2.5 ml) and methanol (0.5 ml). The reaction was stirred for another 18 hours. The mixture was purified by silica column chromatography (0-15% methanol/chloroform) to provide the free base of the title compound as a beige oil (22.3 mg, 25%).

1H NMR (400 MHz, CDCl₃) δ ppm 0.90 (s, 1H) 1.26-1.33 (m, 1H) 1.88 (br. s., 1H) 2.47-2.54 (m, 1H) 2.82 (s, 1H) 2.85 (d, J=3.79 Hz, 1H) 2.97 (d, J=7.83 Hz, 2H) 2.93 (br. s., 1H) 4.00 (br. s., 1H) 4.44 (s, 1H) 4.46 (d, J=1.26 Hz, 1H) 5.32 (s, 5H) 6.63 (s, 1H) 6.65 (s, 1H) 6.89-6.96 (m, 3H) 7.02 (d, J=2.27 Hz, 1H) 7.42 (dd, J=8.59, 4.55 Hz, 1H) 7.69 (d, J=9.35 Hz, 1H).

MS (ES+) m/z 438.

Conversion to the di-HCl salt was accomplished by adding 2 equivalents of 2M HCl in diethyl ether to a DCM solution of the free base of the title compound to provide the title compound as a beige solid (6.0 mg, 6%).

### EXAMPLE 9

### (1R)-9-Fluoro-1-[(4-{[(2E)-3-(5-fluoro-3-pyridinyl)-2-propen-1-yl]amino}-1-piperidinyl)methyl]-1,2-dihydro-4H-pyrrolo[3,2,1-ij]quinolin-4-one dihydrochloride

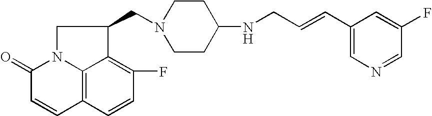

(1R)-1-[(4-Amino-1-piperidinyl)methyl]-9-fluoro-1,2-dihydro-4H-pyrrolo[3,2,1-ij]qin-4-one (for a synthesis see Preparation 1, enantiomer E1) (0.054 g, 0.179 mmol) and (2E)-3-(5-fluoro-3-pyridinyl)-2-propenal (for a synthesis see Preparation 17) (0.027 g, 0.179 mmol) were stirred in DCM (2.5 ml) and methanol (0.5 ml) with sodium sulfate (0.300 g, 2.112 mmol) added to the reaction mixture to facilitate imine formation. After 16.5 hours, sodium triacetoxyborohydride (0.114 g, 0.536 mmol) was added and the mixture stirred for 3.5 hours. The mixture was diluted with DCM and methanol and purified by column chromatography (0-15% methanol/chloroform) to provide the free base of the title compound as a beige, glassy oil (28.4 mg, 36%).

1H NMR (400 MHz, CDCl₃) δ ppm 2.11 (s, 5H) 2.52 (dd, J=12.38, 10.86 Hz, 1H) 2.65 (s, 6H) 2.87 (br. s., 1H) 3.61 (d, J=6.06 Hz, 2H) 4.48 (d, J=3.79 Hz, 1H) 4.50 (d, J=1.26 Hz, 1H) 6.52 (s, 1H) 6.63 (s, 1H) 6.65 (d, J=9.35 Hz, 2H) 6.89 (t, J=8.97 Hz, 1H) 7.42 (dd, J=8.72, 4.42 Hz, 1H) 7.47 (d, J=9.09 Hz, 1H) 7.69 (d, J=9.35 Hz, 1H) 8.37 (d, J=2.53 Hz, 1H) 8.44 (s, 1H).

MS (ES+) m/z 437.

Conversion to the di-HCl salt was accomplished by adding 2 equivalents of 2M HCl in diethyl ether to a DCM solution of the free base of the title compound to provide the title compound as a beigeish-orange (18.9 mg, 21%).

### Example 10

### (1R)-9-Fluoro-1-{[4-({2-[(3-fluorophenyl)oxy]ethyl}amino)-1-piperidinyl]methyl}-1,2-dihydro-4H-pyrrolo[3,2,1-ij]quinolin-4-one dihydrochloride

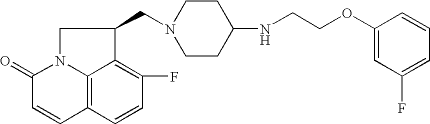

(1R)-1-[(4-Amino-1-piperidinyl)methyl]-9-fluoro-1,2-dihydro-4H-pyrrolo[3,2,1-ij]qin-4-one (for a synthesis see Preparation 1, enantiomer E1) (0.043 g, 0.143 mmol) and [(3-fluorophenyl)oxy]acetaldehyde (for a synthesis see Preparation 18)(0.022 g, 0.143 mmol) were stirred in DCM (2.5 ml) and methanol (0.5 ml) with sodium sulfate (0.300 g, 2.112 mmol) added to the reaction mixture to facilitate imine formation. After 16.5 hours, sodium triacetoxyborohydride (0.091 g, 0.428 mmol) was added and the mixture stirred for 5.25 hours. The mixture was purified by silica column chromatography (0-15% methanol/chloroform) to provide the free base of the title compound as a white, waxy solid (20.9 mg, 33%).

1H NMR (400 MHz, CDCl₃) δ ppm 2.10 (s, 3H) 2.53 (d, J=1.52 Hz, 1H) 2.64 (s, 2H) 2.88 (br. s., 2H) 3.17 (t, J=5.05 Hz, 2H) 4.21 (t, J=5.05 Hz, 2H) 4.48 (s, 1H) 4.49 (d, J=5.56 Hz, 1H) 4.50 (s, 1H) 6.64-6.70 (m, 3H) 6.73 (dd, J=8.46, 2.40 Hz, 1H) 6.89 (t, J=8.97 Hz, 1H) 7.23 (s, 1H) 7.25 (d, J=6.82 Hz, 1H) 7.42 (dd, J=8.72, 4.67 Hz, 1H) 7.69 (d, J=9.60 Hz, 1H)

MS (ES+) m/z 440.

Conversion to the di-HCl salt was accomplished by adding 2 equivalents of 2M HCl in diethyl ether to a DCM solution of the free base of the title compound to provide the title compound as a light yellow solid (14.4 mg, 20%).

### Preparation 1 1-({4-Amino-1-piperidinyl}methyl)-9-fluoro-1,2-dihydro-4H-pyrrolo[3,2,1-ij]quinolin-4-one, (1R)-1-({4-amino-1-piperidinyl}methyl)-9-fluoro-1,2-dihydro-4H-pyrrolo[3,2,1-ij]quinolin-4-one (Enantiomer E1), (1S)-1-({4-amino-1-piperidinyl}methyl)-9-fluoro-1,2-dihydro-4H-pyrrolo[3,2,1-ij]quinolin-4-one (Enantiomer E2)

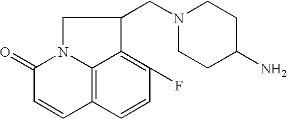

### (a) (2E)-N-(3-Fluoro-2-methylphenyl)-3-phenyl-2-propenamide

A solution of cinnamyl chloride (100 g, 610 mmol) in ethyl acetate (400 ml) was added to a vigorously-stirred mixture of 3-fluoro-2-methylaniline (75 g, 600 mmol), saturated aqueous sodium bicarbonate (850 ml), ice (ca 100 g) and ethyl acetate (400 ml) over 2 min. After 0.25 hour the mixture was filtered, washing with water, more solids coming out of the filtrate and so refiltered. The resulting solid was dried in vacuo (˜160 g, 100%).

MS (+ve ion electrospray) m/z 256 (MH+).

### (b) 7-Fluoro-8-methyl-2(1H)-quinolinone

A slurry of (2E)-N-(3-fluoro-2-methylphenyl)-3-phenyl-2-propenamide (75 g, 305 mmol) in chlorobenzene (400 ml) was treated slowly with aluminium trichloride (163 g, 1.2 mol), with the temperature <30° C. The reaction was stirred vigorously and heated to 65° C. (internal temperature) for 1 hour then to 75° C. (internal temperature) for 0.5 hour. The mixture was allowed to cool (ca 40° C.), then added to excess ice with vigorous stirring. The resulting precipitate was isolated by filtration and washing with water. Drying in vacuo afforded the product (42.5 g, 79%).

MS (+ve ion electrospray) m/z 178 (MH+).

### (c) 7-Fluoro-8-methyl-2-(methyloxy)quinoline

Crude 7-fluoro-8-methyl-2(1H)-quinolinone (46 g, 260 mmol) was suspended in DMSO (300 ml), warmed to 35° C., then treated with potassium t-butoxide (32 g, 286 mmol), under argon (the internal temperature rose to 45° C.). After 15 minutes methyl iodide (21 ml, 48 g, 338 mmol) was added over 2 minutes. (The internal temperature rose to 60° C.). After 30 min the mixture was added to water (2 litres) and extracted with hexane (1.5 litres). The hexane extract was further washed with brine, dried over sodium sulphate, and filtered through a plug of silica, eluting with 1:1 hexane:dichloromethane (500 ml). Evaporation afforded the product (36.8 g, 74%).

MS (+ve ion electrospray) m/z 192 (MH+).

### (d) 8-(Bromomethyl)-7-fluoro-2-(methyloxy)quinoline

A solution of 7-fluoro-8-methyl-2-(methyloxy)quinoline (36.7 g, 192 mmol) in trifluoromethylbenzene (500 ml) was treated with N-bromosuccinimide (37.6 g, 211 mmol) and benzoyl peroxide (243 mg, 1 mmol) and heated at 70° C. (oil bath temperature) while irradiating with a 120 Watt tungsten lamp for 1 hour. The cooled mixture was filtered, washed with dichloromethane, and the combined organic fractions were washed with saturated aqueous sodium bicarbonate solution then dried. The solution was filtered through a plug of silica and evaporated affording a pale yellow solid (51.4 g, 99%).

MS (+ve ion electrospray) m/z 271 (MH+).

### (e) [7-Fluoro-2-(methyloxy)-8-quinolinyl]acetonitrile

A solution 8-(bromomethyl)-7-fluoro-2-(methyloxy)quinoline (22.6 g, 84 mmol) in DMF (600 ml) was treated with potassium cyanide (25 g, 385 mmol) and heated at 70° C. (oil bath temperature) overnight. The mixture was evaporated to dryness and the residue partitioned between ethyl acetate and water. The organic extract was washed with brine, dried and filtered through a plug of silica and evaporated affording a pale brown solid (17. 6 g, 97%).

MS (+ve ion electrospray) m/z 217 (MH+).

### (f) Methyl[7-fluoro-2-(methyloxy)-8-quinolinyl]acetate

(i) A solution of [7-fluoro-2-(methyloxy)-8-quinolinyl]acetonitrile (50 g, 0.231 mol) in dry methanol (850 ml) was treated with trimethylsilyl chloride (100 ml; 0.78 mol) and heated at 79° C. for 2.25 hours. The mixture was evaporated and then partitioned between ethyl acetate (1 L) and water (700 ml). The mixture was filtered to remove methyl(7-fluoro-2-oxo-1,2-dihydro-8-quinolinyl)acetate and the aqueous layer was re-extracted with ethyl acetate (2×300 ml). The combined organic fraction was washed with 2N sodium hydroxide, water (×2), dried (sodium sulphate), and evaporated. The reaction was repeated on the same scale, as above.

The combined reaction products were chromatographed on silica gel (1.5 kg), eluting with dichloromethane, to afford (83.3 g; 72%).

MS (+ve ion electrospray) m/z 250 (MH+).

(ii) The recovered methyl(7-fluoro-2-oxo-1,2-dihydro-8-quinolinyl)acetate (11 g, 46.8 mmol) was suspended in methanol (20 ml), acetonitrile (200 ml) and triethylamine (8 ml, 57 mmol), with stirring together with 2M (trimethylsilyl)diazomethane in hexanes (30 ml, 60 mmol) and the mixture was stirred at room temperature for 3 hours. It was evaporated to dryness and chromatographed on silica gel, eluting with DCM, to afford an additional quantity of methyl[7-fluoro-2-(methyloxy)-8-quinolinyl]acetate (10.8 g). [Total yield 81%]

### (g) Methyl 2-[7-fluoro-2-(methyloxy)-8-quinolinyl]-2-propenoate

A mixture of methyl[7-fluoro-2-(methyloxy)-8-quinolinyl] acetate (48 g; 0.193 mol), paraformaldehyde (41 g; 1.37 mol), potassium carbonate (41 g; 0.295 mol) and benzyltriethyl ammonium chloride (70 g; 0.307 mol) in cyclohexane (1.2 L) was heated at 86° C., with vigorous stirring for 5 hours. The mixture was cooled, water added and the mixture was extracted with ethyl acetate (×3). The mixture was filtered and re-extracted with ethyl acetate (×3). The combined organic fraction was washed with water (×2), brine, and dried. The reaction was repeated on the same scale, as above, and the products combined and evaporated to give a solid (97.9 g; crude yield 97%), sufficiently pure (ca. 90% by NMR) for the next step. The other 10% of material is mainly starting material.

MS (+ve ion electrospray) m/z 262 (MH+).

### (h) Methyl 3-[4-({[(1,1-dimethylethyl)oxy]carbonyl}amino)-1-piperidinyl]-2-[7-fluoro-2-(methyloxy)-8-quinolinyl]propanoate

A solution of methyl 2-[7-fluoro-2-(methyloxy)-8-quinolinyl]-2-propenoate (90% pure; 106 g; equiv. to 0.367 mol), 1,1-dimethylethyl 4-piperidinylcarbamate (80.75 g; 0.404 mol) and 1,1,3,3, tetramethylguanidine (13 ml) in dry DMF (1.2 L) was heated at 80° C. for 2 hours, and then at 50° C. overnight. The mixture was evaporated to dryness, azeotroped with toluene, and chromatographed on silica gel, eluting with hexane and then ethyl acetate-hexane (1:1), affording the product (155.4 g; 92%).

MS (+ve ion electrospray) m/z 462 (MH+).

### (i) 1,1-Dimethylethyl(1-{2-[7-fluoro-2-(methyloxy)-8-quinolinyl]-3-hydroxypropyl}-4-piperidinyl)carbamate

A solution of methyl 3-[4-({[(1,1-dimethylethyl)oxy]carbonyl}amino)-1-piperidinyl]-2-[7-fluoro-2-(methyloxy)-8-quinolinyl]propanoate (76 g, 0.165 mol) in dry tetrahydrofuran (900 ml) at −70° C. was treated with a solution of lithium aluminium hydride in tetrahydrofuran (1M, 196 ml, 0.196 mol) and stirred at this temperature for 1 hour, then at 0-10° C. for 1 hour. Water (18 ml) was cautiously added followed by aqueous sodium hydroxide solution (2M, 33 ml, 0.196 mol), and water (38 ml). The mixture was stirred for 0.5 hour then ether and sodium sulphate were added and the mixture was stirred for 0.5 hour. It was filtered and evaporated, and the residue was recrystallised from ethyl acetate/hexane to give a white solid in two crops (57.7 g; 81%).

MS (+ve ion electrospray) m/z 434 (MH+).

### (j) 1,1-Dimethylethyl{1-[(9-fluoro-4-oxo-1,2-dihydro-4H-pyrrolo[3,2,1-ij]quinolin-1-yl)methyl]-4-piperidinyl}carbamate

A solution of 1,1-dimethylethyl(1-{2-[7-fluoro-2-(methyloxy)-8-quinolinyl]-3-hydroxypropyl}-4-piperidinyl)carbamate (67.35 g, 0.156 mol) in chloroform (1 L) was treated with diisopropylethylamine (60 ml, 0.34 mol) and methanesulphonic anhydride (32.6 g, 0.187 mol). The mixture was stirred at room temperature for 0.5 hour, then heated at 65° C. for 3 hours, and then allowed to cool to room temperature. The mixture was washed with sodium bicarbonate solution (2×1 L), brine (1 L), dried, and evaporated to give a solid (54.75 g; 88%).

MS (+ve ion electrospray) m/z 402 (MH+).

### (k) Title Compound

### Method A

1,1-Dimethylethyl{1-[(9-fluoro-4-oxo-1,2-dihydro-4H-pyrrolo[3,2,1-ij]quinolin-1-yl)methyl]-4-piperidinyl}carbamate (54.75 g, 0.14 mmol) was dissolved in dichloromethane (300 ml) and trifluoroacetic acid (100 ml), stirred at room temperature for 3 hours, evaporated to dryness and azeotroped with toluene. The residue was triturated with ether to give a pink solid that was filtered off, washed with more ether and dried at 35° C. under vacuum overnight to give a solid (62.35 g; 110%—contains excess TFA).

MS (+ve ion electrospray) m/z 302 (MH+).

Racemic material (as trifluoroacetate salt; 114 g) was separated by preparative chiral hplc into the two enantiomers, E1 and E2, using a 20 um Chiralpak AD column, eluting with 80:20:0.1-CH₃CN:CH₃OH:Isopropylamine with Rt E1 7.2 min and Rt E2 8.3 min.

The recovery was E1 29.3 g (97.4% ee) and E2 30.2 g (94.4% ee).

### Method B

1,1-Dimethylethyl{1-[(9-fluoro-4-oxo-1,2-dihydro-4H-pyrrolo[3,2,1-ij]quinolin-1-yl)methyl]-4-piperidinyl}carbamate (92 g, 229 mmol) was treated with concentrated hydrochloric acid (370 ml) and water (300 ml) with external cooling in ice bath. The mixture was stirred overnight, warming to room temperature. The mixture was then concentrated in vacuo at 50° C. for 3 hours. The resultant amorphous gel was triturated with ethanol (1 litre) and stirred vigorously affording a fine white solid. This was isolated by filtration, washing with ether (3×500 ml). Drying in vacuo at 45° C. afforded a white solid (70.56 g, 82%).

1-[(4-Amino-1-piperidinyl)methyl]-9-fluoro-1,2-dihydro-4H-pyrrolo[3,2,1-ij]quinolin-4-one (racemic, dihydrochloride salt) (30 g) was subjected to preparative hplc chromatography on a 20 um Chiralpak AD column eluting with 80:20:0.1 acetonitrile:methanol:isopropylamine affording the E1 enantiomer (Rt 3.6 minutes) as an off-white solid (9.42 g).

Triethylamine can be substituted for isopropylamine in the preparative hplc stage.

### Preparation 2 1-({(3R,4S)-4-Amino-3-hydroxy-1-piperidinyl}methyl)-9-fluoro-1,2-dihydro-4H-pyrrolo[3,2,1-ij]quinolin-4-one

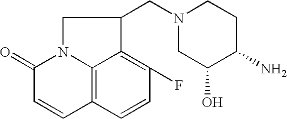

This was prepared from methyl 2-[7-fluoro-2-(methyloxy)-8-quinolinyl]-2-propenoate and 1,1-dimethylethyl[(3R,4S)-3-hydroxy-4-piperidinyl]carbamate (for a synthesis see WO2004058144, Example 34(a) (cis Enantiomer 1)) according to the general method of Preparation 1(h), 1(i), 1(j) and 1(k) affording the product as a white solid, racemic at the benzylic centre.

MS (+ve ion electrospray) m/z 318 (MH+).

### Preparation 3 1-({4-Amino-1-piperidinyl}methyl)-9-fluoro-1-hydroxy-1,2-dihydro-4H-pyrrolo[3,2,1-ij]quinolin-4-one Enantiomers E1 and E2

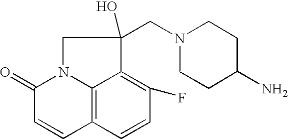

### (a) Methyl 2-[7-fluoro-2-(methyloxy)-8-quinolinyl]-2-oxiranecarboxylate

m-Chloroperbenzoic acid (50%; 6.95 g; 0.0201 mol) was added to a solution of a 1:1 mixture (5.251 g) of methyl 2-[7-fluoro-2-(methyloxy)-8-quinolinyl]-2-propenoate (2.63 g; 0.0101 mol) and methyl[7-fluoro-2-(methyloxy)-8-quinolinyl]acetate in dichloromethane (60 ml) and the mixture was heated at 50° C. for 6.5 hours and then 40° C. until 16 hours. [Further m-chloroperbenzoic acid (3.5 g) was added at 2 hours]. The mixture was cooled, diluted with water and DCM and treated with excess sodium sulfite, followed by aqueous sodium bicarbonate to pH ˜8, and then extracted (3× more) with dichloromethane. The organic fraction was dried, evaporated and chromatographed on silica gel, eluting with 0-100% ethyl acetate-petroleum ether then 0-20% methanol-ethyl acetate to afford the product (2.614 g; 94% based on methyl 2-[7-fluoro-2-(methyloxy)-8-quinolinyl]-2-propenoate starting material).

### (b) 9-Fluoro-1-hydroxy-4-oxo-1,2-dihydro-4H-pyrrolo[3,2,1-ij]quinoline-1-carboxylic acid

A mixture of methyl 2-[7-fluoro-2-(methyloxy)-8-quinolinyl]-2-oxiranecarboxylate (3.105 g; 0.012 mol), and lithium perchlorate (2.38 g; 0.0224 mol) in acetonitrile (30 ml) and water (30 ml) was heated at 85° C. for 120 hours, cooled, and evaporated to dryness. 10% Methanol in dichloromethane was added and the resulting solid was collected and dried to give (1.4 g; 51%).

MS (+ve ion electrospray) m/z 249 (MH+).

### (c) Methyl 9-fluoro-1-hydroxy-4-oxo-1,2-dihydro-4H-pyrrolo[3,2,1-ij]quinoline-1-carboxylate

A solution of 9-fluoro-1-hydroxy-4-oxo-1,2-dihydro-4H-pyrrolo[3,2,1-ij]quinoline-1-carboxylic acid (1.30 g) in methanol (52 ml) was treated with conc. sulphuric acid (0.52 ml) and stirred at room temperature for 1.5 hour. The solution was quenched by stirring with excess MP-carbonate resin until pH ˜7, filtered and evaporated to give a yellow solid (0.855 g; 62%).

MS (+ve ion electrospray) m/z 264 (MH+).

### (d) 9-Fluoro-1-hydroxy-1-(hydroxymethyl)-1,2-dihydro-4H-pyrrolo[3,2,1-ij]quinolin-4-one

A solution of methyl 9-fluoro-1-hydroxy-4-oxo-1,2-dihydro-4H-pyrrolo[3,2,1-ij]quinoline-1-carboxylate (0.855 g; 3.25 mmol) in methanol (85 ml) was cooled to 0° C. and sodium borohydride (0.123 g; 3.25 mmol) added. The mixture was stirred at this temperature for 2 hours. It was quenched with ammonium chloride (5 ml), evaporated to dryness and the residue treated with methanol and then re-evaporated to dryness. Water and dichloromethane were added and the aqueous fraction was evaporated to dryness and again treated with methanol. The resulting solid was filtered off and dried, (0.765 g), sufficiently pure for the next reaction.

MS (+ve ion electrospray) m/z 236 (MH+).

### (c) 9-Fluoro-1-(hydroxymethyl)-4-oxo-1,2-dihydro-4H-pyrrolo[3,2,1-ij]quinolin-1-yl 4-methylbenzenesulfonate

A mixture of 9-fluoro-1-hydroxy-1-(hydroxymethyl)-1,2-dihydro-4H-pyrrolo[3,2,1-ij]quinolin-4-one (0.765 g; 3.25 mmol), p-toluenesulfonyl chloride (0.62 g, 3.25 mmol) and di-n-butyl(oxo)stannane (40.5 mg; 0.1626 mmol) in dichloromethane (30 ml), tetrahydrofuran (30 ml), DMF (3 ml) and triethylamine (0.68 ml) were stirred at room temperature for 16 hours, then sodium bicarbonate solution was added and the mixture was extracted with 10% methanol-dichloromethane. The organic fraction was dried and evaporated to give a yellow oil that was chromatographed on silica gel, eluting with 0-100% ethyl acetate-petroleum ether followed by 0-20% methanol-ethyl acetate to give a yellow oil (0.968 g) (77% yield over 2 steps).

MS (+ve ion electrospray) m/z 390 (MH+).

### (f) 1,1-Dimethylethyl{1-[(9-fluoro-1-hydroxy-4-oxo-1,2-dihydro-4H-pyrrolo[3,2,1-ij]quinolin-1-yl)methyl]-4-piperidinyl}carbamate

A mixture of 9-fluoro-1-(hydroxymethyl)-4-oxo-1,2-dihydro-4H-pyrrolo[3,2,1-ij]quinolin-1-yl 4-methylbenzenesulfonate (0.968 g; 2.49 mmol), 1,1-dimethylethyl 4-piperidinylcarbamate (0.47 g; 2.35 mmol), and anhydrous sodium carbonate (0.746 g; 7.04 mmol), in ethanol (100 ml), was stirred at room temperature for 16 hours. Water was added and the mixture was extracted with 10% methanol-dichloromethane. The organic extracts were dried and evaporated to give a yellow oil (1.038 g; 100%).

MS (+ve ion electrospray) m/z 418 (MH+).

### (g) Title Compounds

A solution of 1,1-dimethylethyl{1-[(9-fluoro-1-hydroxy-4-oxo-1,2-dihydro-4H-pyrrolo[3,2,1-ij]quinolin-1-yl)methyl]-4-piperidinyl}carbamate (1.038 g) in dichloromethane (5 ml) and trifluoroacetic acid (2.5 ml) was stirred at room temperature for 2 hours, during which a further 2 ml trifluoroacetic acid was added, and evaporated to dryness. The residue was dissolved in 1:1 dichloromethane/methanol and stirred with excess MP-carbonate resin until pH ˜8, filtered and evaporated to give a yellow oil

(0.638 g; 81%).

MS (+ve ion electrospray) m/z 318 (MH+).

Racemic material (0.90 g) was separated by preparative chiral hplc into the two enantiomers, E1 and E2, using a Chiralpak AD 10 um (21×250 mm) column, eluting with 80:20:0.1-CH₃CN:CH₃OH:Isopropylamine (20 ml/min) with Rt E1 5.5 min and Rt E2 7.0 min.

The recovery was E1 379 mg (>99% ee) and E2 395 mg (>99% ee).

### Preparation 4 9-Fluoro-1-[((3R)-3-{aminomethyl}-1-pyrrolidinyl)methyl]-1,2-dihydro-4H-pyrrolo[3,2,1-ij]quinolin-4-one

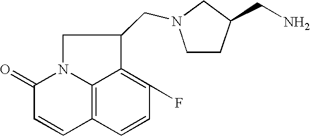

### (a) 1,1-Dimethylethyl(3R)-3-{[(trifluoroacetyl)amino]methyl}-1-pyrrolidinecarboxylate

To a solution of 1,1-dimethylethyl(3R)-3-(aminomethyl)-1-pyrrolidinecarboxylate (2 g, 10 mmol), triethylamine (2.9 ml, 21 mmol) and dimethylaminopyridine (0.13 g, 1 mmol) in DCM (100 ml) was added trifluoroacetic anhydride (1.5 ml, 10.5 mmol) under argon at room temperature. After 2 hours the mixture was treated with water (150 ml) and extracted with 10% methanol in DCM (3×100ml), dried, and the solvent evaporated. The residue was subjected to chromatography on silica gel using 0% -20% methanol-DCM gradient to provide the desired compound (3.12 g, 105%).

δH (CDCl₃, 400 MHz) 1.5 (9H, s), 1.64 (2H, d), 2.04 (1H, m), 2.48 (1H, m), 3.01 (0.5H, m), 3.10 (0.5H, m), 3.20-3.60 (4H, m), 6.50 (0.5H, bs), 6.80 (0.5H, bs).

### (b) 2,2,2-Trifluoro-N-[(35)-3-pyrrolidinylmethyl]acetamide hydrochloride

A solution of 1,1-dimethylethyl(3R)-3-{[(trifluoroacetyl)amino]methyl}-1-pyrrolidinecarboxylate (3.12 g,10 mmol) in DCM (50 ml) was treated slowly with a 4M solution of HCl in dioxane (25 ml). The reaction was stirred at room temperature for 3 hours. The solvent was then removed to afford a pale yellow oil (2.6 g, 112%).

67 H (MeOD, 400 MHz) 1.77 (1H, m), 2.18 (1H, m), 2.64 (1H, m), 2.99 (1H, m), 3.30 (1H, m), 3.70 (5H, m), 9.5 (1H, bs).

### (c) Methyl 2-[7-fluoro-2-(methyloxy)-8-quinolinyl]-3-((3R)-3-{[(trifluoroacetyl)amino]methyl}-1-pyrrolidinyl)propanoate

A solution of methyl 2-[7-fluoro-2-(methyloxy)-8-quinolinyl]-2-propenoate (2.4 g, 9.2 mmol), 2,2,2-trifluoro-N-[(35)-3-pyrrolidinylmethyl]acetamide hydrochloride (2.4 g, 10.12 mmol) and triethylamine (3.4 ml, 23 mmol) in DMF (30 ml) was stirred and heated at 60° C. overnight. The solvent was removed in vacuo and the residue was subjected to chromatography on silica gel using a 0%-10% methanol-DCM gradient to give a brown oil (4.2 g, 100%).

MS (+ve ion electrospray) m/z 458 (MH+).

### (d) Methyl 3-[(3R)-3-(aminomethyl)-1-pyrrolidinyl]-2-[7-fluoro-2-(methyloxy)-8-quinolinyl]propanoate

Methyl 2-[7-fluoro-2-(methyloxy)-8-quinolinyl]-3-((3R)-3-{[(trifluoroacetyl)amino]methyl}-1-pyrrolidinyl)propanoate (3.4 g, 7.4 mmol) was treated with a 7% solution of potassium carbonate in 2:5 water:methanol (119 ml) for 4 hours. The solvents were then evaporated and the residue redissolved in 20% methanol in DCM. The organic phase was dried over magnesium sulphate and the solvent was removed under reduced pressure. The residue was subjected to chromatography on silica gel using a gradient of 0-20% 2M ammonia-methanol in DCM to provide the desired compound (2.2 g, 82%).

MS (+ve ion electrospray) m/z 362 (MH+).

### (e) Methyl 3-{(3R)-3-[({[(1,1-dimethylethyl)oxy]carbonyl}amino)methyl]-1-pyrrolidinyl}-2-[7-fluoro-2-(methyloxy)-8-quinolinyl]propanoate

A solution of methyl 3-[(3R)-3-(aminomethyl)-1-pyrrolidinyl]-2-[7-fluoro-2-(methyloxy)-8-quinolinyl]propanoate (2.2 g, 6.1 mmol) and triethylamine (0.86 ml, 6.1 mmol) in DCM (30 ml) was treated with a solution of di-tert-butyl dicarbonate (1.3 g, 6.1 mmol) in DCM at 0° C. After stirring the mixture at room temperature for 1 hour, water (50 ml) was added and the aqueous fraction was extracted with 20% methanol in DCM (3×200 ml). The organic phase was dried and the solvent was evaporated. The residue was subjected to chromatography on silica gel using a 0-10% methanol-DCM gradient to provide the desired compound (2.56 g, 87%).

MS (+ve ion electrospray) m/z 462 (MH+).

### (f) 1,1-Dimethylethyl[((3R)-1-{2-[7-fluoro-2-(methyloxy)-8-quinolinyl]-3-hydroxypropyl}-3-pyrrolidinyl)methyl]carbamate

A solution of methyl 3-{(3R)-3-[({[(1,1-dimethylethyl)oxy]carbonyl}amino)methyl]-1-pyrrolidinyl}-2-[7-fluoro-2-(methyloxy)-8-quinolinyl]propanoate (2.56 g, 5.55 mol) in dry tetrahydrofuran (60 ml) at −78° C. under argon was treated with a solution of lithium aluminium hydride in tetrahydrofuran (1 M, 7.2 ml, 7.2 mmol) and then slowly allowed to warm to room temperature. After 0.5 hour, water (0.5 ml) was added followed by aqueous sodium hydroxide solution (2 M, 0.9 ml) and water (1 ml). The mixture was stirred at ambient temperature for 1 hour. It was filtered and evaporated, and the residue was subjected to chromatography on silica gel using a 0-20% methanol-DCM gradient to provide the desired compound (1.88 g, 78%).

MS (+ve ion electrospray) m/z 434 (MH+).

### (g) 1,1-Dimethylethyl({(3R)-1-[(9-fluoro-4-oxo-1,2-dihydro-4H-pyrrolo[3,2,1-ij]quinolin-1-yl)methyl]-3-pyrrolidinyl}methyl)carbamate

A solution of 1,1-dimethylethyl[((3R)-1-{2-[7-fluoro-2-(methyloxy)-8-quinolinyl]-3-hydroxypropyl}-3-pyrrolidinyl)methyl]carbamate (1.88 g, 4.4 mmol) in chloroform (20 ml) was treated with diisopropylethylamine (1.2 ml, 7.04 mmol) and methanesulphonyl chloride (0.45 ml, 5.5 mmol) at 0° C. under argon. The mixture was stirred at 0° C. for 0.5 hour, warmed to rt and stirred for 1 hour then heated at 45° C. overnight, and allowed to cool to room temperature. The mixture was diluted with DCM and washed with sodium bicarbonate solution. The aqueous was extracted with 10% methanol in DCM (3×80 ml). The organic phase was dried and the solvent evaporated. The residue was subjected to chromatography on silica gel using a 0-10% methanol-DCM gradient to provide the desired compound (1.47 g, 84%). MS (+ve ion electrospray) m/z 402 (MH+).

### (h) Title Compound

1,1-Dimethylethyl({(3R)-1-[(9-fluoro-4-oxo-1,2-dihydro-4H-pyrrolo[3,2,1-ij]quinolin-1-yl)methyl]-3-pyrrolidinyl}methyl)carbamate (1.47 g, 3.7 mmol) was dissolved in dichloromethane (15 ml) and trifluoroacetic acid (15 ml) and stirred at room temperature for 30 minutes, then evaporated to dryness. The residue was redissolved in methanol and stirred with excess Amberlyst® A21 ion-exchange resin (Aldrich: a weakly basic, macroreticular resin with alkyl amine functionality) for 1 hour and then filtered. The solvent was removed under reduced pressure and the residue was subjected to chromatography on silica gel using a 0-20% 2M ammonia in methanol-DCM gradient to provide the desired compound (0.75 g, 68%)

MS (+ve ion electrospray) m/z 302 (MH+).

### Preparation 5 1-[(4-amino-1-piperidinyl)methyl]-1,2-dihydro-4H-pyrrolo[3,2,1-ij]quinolin-4-one dihydrochloride

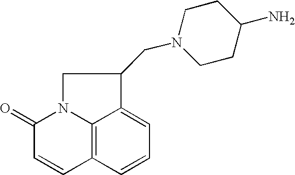

### (a) (2E)-N-(2-Bromophenyl)-3-phenyl-2-propenamide

To a solution of 2-bromoaniline (22.27 g, 0.13 mol) and potassium carbonate (26.8 g, 0.13 mol) in acetone (50 ml) and water (65 ml) at 0° C. was added cinnamoyl chloride (21.57 g, 0.13 mol) portionwise over 15 minutes. Another 150 ml of both acetone and water was then added to facilitate stirring. The reaction was stirred for 2 hours at 0° C. before being added to ice water (400 ml). The resultant solid was filtered, washed with water (500 ml) and dried in vacuo. The resultant solid was triturated with hot hexane and dried in vacuo to provide the desired compound as a white solid (29.50 g, 75%).

MS (ES+) m/z 303 (MH⁺, 100%).

### (b) 8-Bromo-2(1H)-quinolinone

To a suspension of (2E)-N-(2-bromophenyl)-3-phenyl-2-propenamide (22.9 g, 76.0 mol) in chlorobenzene (100 ml) under an argon atmosphere at room temperature was added aluminium trichloride (60.78 g, 133.34 mmol). The reaction was heated for 2 hours at 125° C. after which time the reaction mixture was cooled to 50° C. before being carefully added to ice water (3 L). The resultant solid was filtered and then washed with water (500 ml), then triturated with hot ethanol, filtered and dried in vacuo to provide the desired compound as a white solid (7.39 g, 75%).

MS (ES+) m/z 225 (MH⁺, 100%).

### (c) 8-Bromo-2-(methyloxy)quinoline

To a suspension of 8-bromo-2(1H)-quinolinone (2.76 g, 12.32 mmol) in N,N-dimethylformamide (40 ml) under an argon atmosphere at 0° C. was added potassium carbonate (3.4 g, 24.63 mmol). The reaction was then stirred for 15 minutes before methyl iodide (0.91 ml, 14.78 mmol) was added. The reaction was allowed to warm to room temperature and then stirred for 3 hours. The reaction mixture was then evaporated and the residue treated with dichloromethane and water. The aqueous fraction was re-extracted with dichloromethane. The combined organic fractions were then dried (MgSO₄), the solvent was removed under reduced pressure and then the residue was subjected to chromatography on silica gel using a methanol-dichloromethane gradient. This provided the desired compound as a yellow solid (2.16 g, 74%).

MS (ES+) m/z 239 (MH⁺, 100%).

### (d) [2-(Methyloxy)-8-quinolinyl]boronic acid

According to the literature procedure (Li, W.; Nelson, D.; Jensen, M.; Hoerrner, R.; Cai, D.; Larsen, R.; Reider, P J. Org. Chem. (2002), 67(15), 5394) a solution of 8-bromo-2-(methyloxy)quinoline (1.95 g, 8.19 mmol) and triisopropylborate (2.30 ml, 9.83 mmol) in toluene (20 ml) and tetrahydrofuran (5 ml) under an argon atmosphere was cooled to −78° C. A solution of n-butyl lithium (2.5M in hexanes, 3.9 ml, 9.83 mmol) was then added dropwise over 20 minutes. The reaction was stirred at −78° C. for 2 hours and then warmed to −20° C. The reaction was then quenched with 2M HCl solution (10 ml) and treated with dichloromethane. The aqueous fraction was re-extracted with dichloromethane. The combined organic fractions were then dried (MgSO₄) and the solvent removed under reduced pressure. The residue was triturated with hexane to give the desired compound as a yellow solid (453 mg, 40%).

MS (ES+) m/z 204 (MH⁺, 100%).

### (e) Methyl 2-[2-(Methyloxy)-8-quinolinyl]-2-propenoate

To a solution of methyl 2-bromo-2-propenoate (452 mg, 2.74 mmol) (for a synthesis see Rachon, J.; Goedken, V.; Walborsky, H. J. Org. Chem. (1989), 54(5), 1006) in degassed tetrahydrofuran (10 ml) under an argon atmosphere was added [2-(methyloxy)-8-quinolinyl]boronic acid (506 mg, 2.49 mmol), bis(tri-t-butylphosphine)palladium (0) (25 mg, 0.05 mmol), bis(dibenzylideneacetone)palladium(0) (23 mg, 0.025 mmol) and potassium fluoride (477 mg, 8.217 mmol). The reaction was heated at 70° C. for 24 hours and then treated with water and dichloromethane. The aqueous fraction was re-extracted with dichloromethane. The combined organic fractions were then dried (MgSO₄) and the solvent removed under reduced pressure. The residue was subjected to chromatography on silica gel using a ethyl acetate-hexane gradient. This provided the desired compound as a yellow solid (381 mg, 63%).

MS (ES+) m/z 244 (MH⁺, 100%), 212 (80%).

### (f) Methyl 3-[4-({[(1,1-Dimethylethyl)oxy]carbonyl}amino)-1-piperidinyl]-2-[2-(methyloxy)-8-quinolinyl]propanoate

To a solution of methyl 2-[2-(methyloxy)-8-quinolinyl]-2-propenoate (381 mg, 1.57 mmol) in N,N′-dimethylformamide (5 ml) and tetramethylguanidine (0.05 ml) was added 1,1-dimethylethyl 4-piperidinylcarbamate (345 mg, 1.73 mmol). The reaction mixture was stirred for 12 hour at 60° C. after which time the solvent was removed under reduced pressure. The residue was subjected to chromatography on silica gel using a methanol-dichloromethane gradient. This provided the desired compound as a yellow solid (546 mg, 79%).

MS (ES+) m/z 444 (MH⁺, 100%).

### (g) 1,1-Dimethylethyl(1-{3-hydroxy-2-[2-(methyloxy)-8-quinolinyl]propyl}-4-piperidinyl)carbamate

To a solution of methyl 3-[4-({[(1,1-dimethylethyl)oxy]carbonyl}amino)-1-piperidinyl]-2-[2-(methyloxy)-8-quinolinyl]propanoate (546 mg, 1.23 mmol) in tetrahydrofuran (20 ml) at −78° C. was added lithium aluminium hydride (1M in tetrahydrofuran, 1.50 ml, 1.48 mmol). The reaction was then stirred at −78° C. for 0.5 hours before water (0.2 ml) and then 2M NaOH solution (0.4 ml) was added and the mixture warmed to 25° C. The mixture was then filtered, dried (MgSO₄) and the solvent was removed under reduced pressure. The residue was subjected to chromatography on silica gel using a methanol-dichloromethane gradient. This provided the desired compound as a white solid (370 mg, 72%).

MS (ES+) m/z 416 (MH⁺, 100%).

### (h) 1,1-Dimethylethyl{1-[(4-oxo-1,2-dihydro-4H-pyrrolo[3,2,1-ij]quinolin-1-yl)methyl]-4-piperidinyl}carbamate

To a solution of 1,1-dimethylethyl(1-{3-hydroxy-2-[2-(methyloxy)-8-quinolinyl]propyl}-4-piperidinyl)carbamate (370 mg, 0.892 mmol) in chloroform (20 ml) at 0° C. was added diisopropylethylamine (0.33 ml, 1.96 mmol) and methanesulfonic anhydride (0.186 g, 1.07 mmol). The reaction was then heated at 70° C. for 5 hours and then treated with dichloromethane and water. The aqueous phase was extracted twice with dichloromethane and the combined organic phases were dried (MgSO₄) and the solvent was removed under reduced pressure. The residue was subjected to chromatography on silica gel using a methanol-dichloromethane gradient to provide the desired compound (0.276 g, 81%).

MS (ES+) m/z 384 (MH⁺, 10%), 284 (100%).

### (i) Title Compound

A solution of 1,1-dimethylethyl{1-[(4-oxo-1,2-dihydro-4H-pyrrolo[3,2,1-ij]quinolin-1-yl)methyl]-4-piperidinyl}carbamate (276 mg, 0.721 mmol) in chloroform (5 ml) and MeOH (5 ml) was treated with 4M HCl in dioxane (10 ml) and stirred at room temperature for 2 hours. The reaction mixture was evaporated to provide the desired compound (0.283 g, 110%) as the slightly impure dihydrochloride salt which was used without further purification.

MS (ES+) m/z 306 (M+Na, 10%), 284 (MH+, 100%).

### Preparation 6 1-({4-amino-1-piperidinyl}methyl)-4-oxo-1,2-dihydro-4H-pyrrolo[3,2,1-ij]quinoline-9-carbonitrile

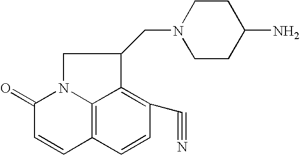

### (a) Methyl 2-[7-bromo-2-(methyloxy)-8-quinolinyl]-3-[4-({[(1,1-dimethylethyl)oxy]carbonyl}amino)-1-piperidinyl]propanoate

A solution of methyl 2-[7-bromo-2-(methyloxy)-8-quinolinyl]-2-propenoate (12.4 g, 38.5 mmol), 1,1-dimethylethyl 4-piperidinylcarbamate (8.5 g, 42.3 mmol) and 1,1,3,3, tetramethylguanidine (10 drops) in dry DMF (120 mL) was heated at 70° C. for 3 days. More 1,1-dimethylethyl 4-piperidinylcarbamate (1.5 g) was added and the mixture heated at 100° C. for a further day. The mixture was evaporated and the residue chromatographed on silica eluting with 2% methanol in dichloromethane affording a pale yellow solid (17.1 g, 85%).

MS (+ve ion electrospray) m/z 523 (MH+).

### (b) 1,1-Dimethylethyl(1-{2-[7-bromo-2-(methyloxy)-8-quinolinyl]-3-hydroxypropyl}-4-piperidinyl)carbamate

A solution of methyl 2-[7-bromo-2-(methyloxy)-8-quinolinyl]-3-[4-({[(1,1-dimethylethyl)oxy]carbonyl}amino)-1-piperidinyl]propanoate (17 g, 32.5 mmol) in THF (300 mL) at −78° C. under argon was treated with a solution of lithium aluminium hydride in THF (1M, 39 mL, 39 mmol). The reaction was stirred at −78° C. for 1 hour then allowed to stir at room temperature for 2 hours. Water (18 mL) was added followed by aqueous sodium hydroxide solution (2M, 40 mL) and more water (20 mL). Filtration and evaporation afforded a solid. This was chromatographed eluting with 0-20% methanol in dichloromethane affording a yellow solid (9.9 g, 61%).

MS (+ve ion electrospray) m/z 495 (MH+).

### (c) 1,1-Dimethylethyl{1-[(9-bromo-4-oxo-1,2-dihydro-4H-pyrrolo[3,2,1-ij]quinolin-1-yl)methyl]-4-piperidinyl}carbamate

A solution of 1,1-dimethylethyl(1-{2-[7-bromo-2-(methyloxy)-8-quinolinyl]-3-hydroxypropyl}-4-piperidinyl)carbamate (9.9 g, 20 mmol), methanesulphonic anhydride (4.2 g, 24 mmol) and diisopropylethylamine (7.7 mL, 44 mmol) in chloroform (260 mL) was heated at 60° C. (oil bath temperature) for 1 hour, then heated to reflux for 1.5 hours. The mixture was evaporated and the residue chromatographed eluting with 0-30% methanol in ethyl acetate affording a white solid (4.7 g, 51%).

MS (+ve ion electrospray) m/z 463 (MH+).

### (d) 1,1-Dimethylethyl{1-[(9-cyano-4-oxo-1,2-dihydro-4H-pyrrolo[3,2,1-ij]quinolin-1-yl)methyl]-4-piperidinyl}carbamate

A mixture of 1,1-dimethylethyl{1-[(9-bromo-4-oxo-1,2-dihydro-4H-pyrrolo[3,2,1-ij]quinolin-1-yl)methyl]-4-piperidinyl}carbamate (4.7 g, 10.2 mmol), copper(I) cyanide (3.3 g, 36.6 mmol) and DMF (60 mL) was heated at 135° C. for 2 hours. The mixture was evaporated to dryness and the residue partitioned between saturated aqueous ammonia and dichloromethane. The aqueous phase was further extracted with dichloromethane and the combined organic extracts dried and evaporated (3.2 g). The aqueous phase was further extracted twice with ethyl acetate and these extracts were combined, dried and evaporated (0.5 g). The residues (3.7 g in total) were combined and chromatographed eluting with 0-15% methanol in ethyl acetate affording a white solid (2.7 g, 65%).

MS (+ve ion electrospray) m/z 409 (MH+).

### (e) 1-[(4-Amino-1-piperidinyl)methyl]-4-oxo-1,2-dihydro-4H-pyrrolo[3,2,1-ij]quinoline-9-carbonitrile

A solution of 1,1-dimethylethyl{1-[(9-cyano-4-oxo-1,2-dihydro-4H-pyrrolo[3,2,1-ij]quinolin-1-yl)methyl]-4-piperidinyl}carbamate (2.65 g, 6.5 mmol) in dichloromethane (50 mL), was treated with TFA (50 mL). After 30 minutes the mixture was evaporated and the residue was twice azeotroped with chloroform then triturated with ether (three times). The resulting solid was redissolved in dichloromethane/methanol (60 mL/120 mL) and treated with MP-carbonate resin (3 mmol of carbonate per gramme, 22 g, 66 mmol). The resin was removed by filtration, washing with dichloromethane and methanol. Evaporation of the filtrate afforded a white solid (2 g)

MS (+ve ion electrospray) m/z 309 (MH+).

### Preparation 7 1-({4-Amino-1-piperidinyl}methyl)-9-(methyloxy)-1,2-dihydro-4H-pyrrolo[3,2,1-ij]quinolin-4-one

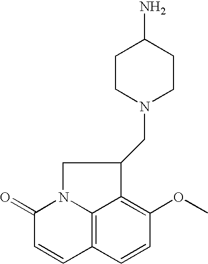

A solution of 1-[(4-amino-1-piperidinyl)methyl]-9-fluoro-1,2-dihydro-4H-pyrrolo[3,2,1-ij]quinolin-4-one dihydrochloride (1.74 g, 4.64 mmol) in methanol (17 mL) at room temperature under argon, was treated with 25% sodium methoxide in methanol (2.5 ml, 11.5 mmol). The reaction was then heated to 85° C. for 2 hours, then a further addition of 25% sodium methoxide in methanol (5 ml) was added. The reaction was left at 85° C. overnight (16 hours). A further addition of 25% sodium methoxide in methanol (5 ml) was required in the morning and reaction was left at 85° C. for most of the day. The reaction mixture was treated with ammonium chloride (saturated) until the pH reached 8, where the solvent was removed under vacuum. The residue was re-dissolved in 10% methanol in DCM and stirred at room temperature for 1 hour with sodium sulphate. This mixture was then filtered and the solvent removed to give a yellow solid (3.81 g). This was purified on a 10 g SCX column eluting with methanol then 2M ammonia in methanol to give a yellow oily solid (0.832, 57%).

MS (ES+) m/z 314 (MH⁺).

### Preparation 8 (4R/S)-3-Chloro-4-({4-amino-1-piperidinyl}methyl)-4-hydroxy-4,5-dihydro-7H-pyrrolo[3,2,1-de]-1,5-nanphthyridin-7-one

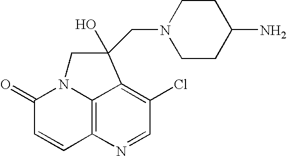

### (a) Dimethyl[3-chloro-6-(methyloxy)-1,5-naphthyridin-4-yl]propanedioate

### Method 1

To a solution of dimethylmalonate (82 ml, 715 mmol) in N,N-dimethylformamide (1400 ml) at 0° C. was added sodium hydride (60% dispersion in oil)(28.6 g, 715 mmol). The mixture was stirred for 0.5 hour and sonicated for 0.5 hour before adding 1,1,1-trifluoro-methanesulfonic acid 3-chloro-6-methoxy-[1,5]naphthyridin-4-yl ester (for a synthesis see WO2004058144, Example 1(b)) (88.88 g, 238.5 mmol). The reaction mixture was then heated at 50° C. for 12 hours. The reaction was cooled, treated with ethyl acetate, water and HCl (2M) (340 ml). The organic phase was washed twice with water, dried and the solvent was removed under reduced pressure. The residue was treated with toluene and stirred for 1 hour. Filtration gave the desired compound. The toluene solution was subjected to column chromatography on silica gel using a hexane and ethyl acetate gradient to provide more of the desired compound; (total yield: 62.72 g, 81%).

MS (+ve ion electrospray) m/z 325 (MH⁺).

### Method 2

### (i) 8-Bromo-7-chloro-2-(methyloxy)-1,5-naphthyridine

To a solution of 3-chloro-6-(methyloxy)-1,5-naphthyridin-4(1H)-one (3-chloro-6-(methoxy)-1,5-naphthyridin-4-ol, for a synthesis see WO2004058144, Example 1(a)) (43 g, 204.8 mmol) in N,N-dimethylformamide (500 ml) at 0° C. was added phosphorous tribromide dropwise (23.3 ml, 245.8 mmol). The reaction mixture was then stirred at 10° C. for 0.5 hour and then at 25° C. for 1 hour. The mixture was then poured onto 1600 ml of water and basified to pH 7 with potassium carbonate. The solid formed was filtered off, washed with water and dried in vacuo to afford the desired compound (51 g, 91%).

MS (+ve ion electrospray) m/z 274 (MH⁺)

(ii) Dimethyl[3-chloro-6-(methyloxy)-1,5-naphthyridin-4-yl]propanedioate To a solution of dimethylmalonate (68.6 ml, 600 mmol) in 1,4-dioxane (600 ml) was added sodium hydride (60% dispersion in oil)(22 g, 550 mmol). The mixture was stirred at 75° C. for 2 hours before adding 8-bromo-7-chloro-2-(methyloxy)-1,5-naphthyridine (54.5 g, 200 mmol) and copper(I) bromide (10 g, 69.7 mmol). The reaction mixture was then heated at 100° C. for 12 hours. The reaction was cooled, treated with ethyl acetate, water and HCl (2N) (175 ml). The aqueous phase was extracted with ethyl acetate. The organic phase was washed twice with water, dried and the solvent was removed under reduced pressure. The residue was subjected to column chromatography on silica gel using a hexane and ethyl acetate gradient to provide the desired compound (63 g, 97%). MS (+ve ion electrospray) m/z 325 (MH⁺).

### (b) Methyl[3-chloro-6-(methyloxy)-1,5-naphthyridin-4-yl]acetate

To a solution of dimethyl[3-chloro-6-(methyloxy)-1,5-naphthyridin-4-yl]propanedioate (56 g, 173 mmol) in dimethylsulfoxide (1210 ml) was added lithium chloride (14.9 g, 350 mmol) and water (3.2 ml, 180 mmol). The mixture was heated to 100° C. for 16 hours then cooled and treated with ethyl acetate and water. The organic phase was washed twice with water, the aqueous extracted with ethyl acetate and this water-washed. The combined organic phases were dried and the solvent was removed under reduced pressure. The residue was subjected to column chromatography on silica gel using a hexane and ethyl acetate gradient to provide the desired compound (43 g, 94%).

MS (+ve ion electrospray) m/z 267 (MH⁺).

### (c) Methyl 2-[3-chloro-6-(methyloxy)-1,5-naphthyridin-4-yl]-2-propenoate

To a solution of methyl[3-chloro-6-(methyloxy)-1,5-naphthyridin-4-yl]acetate (43 g, 162 mmol) in cyclohexane (1060 ml)was added benzyltriethylammonium chloride (71.2 g, 313 mmol), potassium carbonate (42 g, 304 mmol) and paraformaldehyde (42 g). The reaction mixture was then heated at 80° C. for 24 hours, cooled and treated with ethyl acetate and water. The aqueous was extracted with ethyl acetate. The combined organic phases were dried and the solvent was removed under reduced pressure. The residue was subjected to column chromatography on silica gel using a hexane and ethyl acetate gradient to provide the desired compound (40 g, 89%).

MS (+ve ion electrospray) m/z 279 (MH⁺).

### (d) Methyl(2R/S)-2-[3-chloro-6-(methyloxy)-1,5-naphthyridin-4-yl]-3-[(3R,4S)-4-({[(1,1-dimethylethyl)oxy]carbonyl}amino-3-hydroxy-1-piperidinyl}propanoate

A mixture of methyl 2-[3-chloro-6-(methyloxy)-1,5-naphthyridin-4-yl]-2-propenoate (10 g), 1,1-dimethylethyl[(3R,4S)-3-hydroxy-4-piperidinyl]carbamate (for a synthesis, see WO2004058144, Example 34(a), cis-4-tert-butoxycarbonylamino-3-hydroxy-piperidine enantiomer 1) (8.05 g) and 1,1,3,3-tetramethylguanidine (1 ml) in N,N-dimethylformamide (40 ml) was heated at 80° C. for 4 hours, cooled and evaporated to dryness. Chromatography, eluting with methanol/dichloromethane, gave the product (17.4 g 98%).

MS (+ve ion electrospray) m/z 495 (MH⁺).

### (c) 1,1-Dimethylethyl((3R,4S)-1-{(2R/S)-2-[3-chloro-6-(methyloxy)-1,5-naphthyridin-4-yl]-3-hydroxypropyl}-3-hydroxy-4-piperidinyl)carbamate

A solution of methyl(2R/S)-2-[3-chloro-6-(methyloxy)-1,5-naphthyridin-4-yl]-3-[(3R,4S)-4-{[(1,1-dimethylethyl)oxy]carbonyl}amino-3-hydroxy-1-piperidinyl}propanoate(17.4 g, 35.2 mmol) in THF (400 ml) at −70° C. under argon was treated dropwise with a 1M solution of lithium aluminium hydride in THF (40.5 ml, 40.5 mmol) and allowed to warm gradually to 0° C. The solution was stirred at this temperature for 2 hours, treated with water (3 ml), 2N sodium hydroxide (5.7 ml) and water (6.6 ml), stirred for 1 hour at room temperature and filtered. The filtrate was evaporated and the residue chromatographed, eluting with methanol/dichloromethane to give the product (8.75 g, 53%).

MS (+ve ion electrospray) m/z 467 (MH⁺).

### (f) 3-Chloro-4-methylidene-4,5-dihydro-7H-pyrrolo[3,2,1-de]-1,5-naphthyridin-7-one

A solution of 1,1-dimethylethyl((3R,4S)-1-{(2R/S)-2-[3-chloro-6-(methyloxy)-1,5-naphthyridin-4-yl]-3-hydroxypropyl}-3-hydroxy-4-piperidinyl)carbamate (8.75 g, 18.7 mmol) and diisopropylethylamine (4.9 ml) in dichloromethane (90 ml) was treated at 0° C. under argon with para-toluenesulphonic anhydride (6.7 g, 20.6 mmol). After 3 hours at room temperature more para-toluenesulphonic anhydride (0.7 g) was added. After 2.5 days the mixture was washed with saturated aqueous sodium bicarbonate solution. The aqueous phase was extracted with dichloromethane (2×) and the combined organic extracts dried and evaporated. The residue was chromatographed, eluting with methanol/dichloromethane to give firstly the partially purified title compound (0.84 g, 21%) then 1,1-dimethylethyl{(3R,4S)-1-[(3-chloro-7-oxo-4,5-dihydro-7H-pyrrolo[3,2,1-de]-1,5-naphthyridin-4-yl)methyl]-3-hydroxy-4-piperidinyl}carbamate (3.93 g, 48%). The partially purified title compound (0.84 g) was further purified by chromatography eluting with methanol/dichloromethane affording pure product (0.57 g).

MS (+ve ion electrospray) m/z 219 (MH+).

### (g) (4R/S)-3-Chloro-4-hydroxy-4-(hydroxymethyl)-4,5-dihydro-7H-pyrrolo[3,2,1-de]-1,5-naphthyridin-7-one

A solution of 3-chloro-4-methylidene-4,5-dihydro-7H-pyrrolo[3,2,1-de]-1,5-naphthyridin-7-one (565 mg, 2.6 mol) in tent-butanol/water (25 ml/25 ml) was treated with AD-mix alpha/beta (3.5 g/3.5 g) and stirred overnight. Sodium sulphite (5 g) was added and the mixture was stirred for 1 hour. Ethyl acetate, saturated aqueous sodium bicarbonate solution and brine were added. The phases were separated and the aqueous phase was extracted twice more with ethyl acetate. The combined organic extracts were dried and evaporated. The crude material was chromatographed affording the product (300 mg).

MS (+ve ion electrospray) m/z 253 (MH+).

### (h) R4R/S)-(3-Chloro-4-hydroxy-7-oxo-4,5-dihydro-7H-pyrrolo[3,2,1-de]-1,5-naphthyridin-4-yl)]methyl 4-methylbenzenesulfonate

A suspension of (4R/S)-3-chloro-4-hydroxy-4-(hydroxymethyl)-4,5-dihydro-7H-pyrrolo[3,2,1-de]-1,5-naphthyridin-7-one (300 mg, 1.2 mmol) in dichloromethane/tetrahydrofuran/N,N-dimethylformamide (10 ml/10 ml/2 ml) was treated with triethylamine (0.25 ml, 1.8 mmol), para-toluenesulphonyl chloride (220 mg, 1.2 mol) and dibutyltin oxide (15 mg, 0.06 mmol). After 3 hours water was added and the phases separated. The organic phase was washed with saturated aqueous sodium bicarbonate solution then dried and evaporated affording the product (465 mg).

MS (+ve ion electrospray) m/z 407 (MH+).

### (i) 1,1-dimethylethyl(1-{[(4R/S)-3-chloro-4-hydroxy-7-oxo-4,5-dihydro-7H-pyrrolo[3,2,1-de]-1,5-naphthyridin-4-yl]methyl}-4-piperidinyl)carbamate

A mixture of R4R/S)-(3-chloro-4-hydroxy-7-oxo-4,5-dihydro-7H-pyrrolo[3,2,1-de]-1,5-naphthyridin-4-yl)methyl 4-methylbenzenesulfonate (465 mg, 1.15 mmol), 1,1-dimethylethyl 4-piperidinylcarbamate (218 mg, 1.1 mmol) and sodium carbonate (350 mg, 3.3 mmol) in ethanol (12.5 ml). After 1 day more 1,1-dimethylethyl 4-piperidinylcarbamate (100 mg) was added. After 1 hour the mixture was evaporated and the residue was partitioned between 20% methanol/dichloromethane and water. The organic extract was dried and evaporated. The crude material was chromatographed affording the product (410 mg, 82%).

MS (+ve ion electrospray) m/z 435.5 (MH+).

### (j) Title Compound

1,1-dimethylethyl(1-{[(4R/S)-3-chloro-4-hydroxy-7-oxo-4,5-dihydro-7H-pyrrolo[3,2,1-de]-1,5-naphthyridin-4-yl]methyl}-4-piperidinyl)carbamate (410 mg) was dissolved in dichloromethane/trifluoroacetic acid (5 ml/4 ml). After 30 minutes the mixture was evaporated. The residue was dissolved in methanol and treated with Amberlyst resin. After 1 hour the mixture was filtered and the filtrate evaporated to dryness. The crude material was chromatographed on silica eluting with 0-20% of 2M ammonia/methanol in dichloromethane affording the product (300 mg, 95%).

MS (+ve ion electrospray) m/z 335.5 (MH+).

### Preparation 9 3,8-Difluoro-4-hydroxy-4-({4-amino-1-piperidinyl}methyl)-4,5-dihydro-7H-pyrrolo[3,2,1-de]-1,5-naphthyridin-7-one

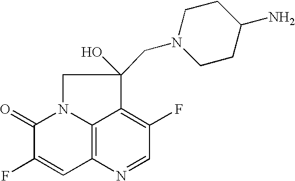

### (a) 2-Chloro-5-fluoro-6-(methyloxy)-3-pyridinecarboxylic acid

A solution of 2,6-dichloro-5-fluoro-3-pyridinecarboxylic acid (51.12 g, 243 mmol) in methanol (400 ml) was treated with sodium methoxide in methanol (25% w/v, 100 ml, 535 mmol) and the mixture heated to reflux for 4 hours. The cooled mixture was treated with water (400 ml) and acidified to pH2 with aqueous hydrochloric acid (2M) then concentrated to ca 400 ml. Filtration, washing with water and drying in vacuo over P₂O₅ for 18 h afforded the product as a white solid (32.65 g, 65%).

MS (+ve ion electrospray) m/z 208 (MH+).

### (b) 1,1-Dimethylethyl[2-chloro-5-fluoro-6-(methyloxy)-3-pyridinyl]carbamate

A mixture of 2-chloro-5-fluoro-6-(methyloxy)-3-pyridinecarboxylic acid (32.65 g, 159 mmol) in toluene/triethylamine/tert-butanol (300 ml/26.4 ml/75 ml) was treated with diphenylphosphoryl azide (37.7 ml, 174.8 mmol) and heated to 100° C. for 2 hours. The mixture was treated with saturated aqueous sodium bicarbonate solution (500 ml) then extracted with ethyl acetate (3×500 ml). The combined organic extracts were dried over magnesium sulphate and evaporated. The residue was chromatographed eluting with 0-40% dichloromethane in petrol affording the product as a white solid (36.35 g, 83%).

MS (+ve ion electrospray) m/z 221/223 (M(-t-Bu)H+).

### (c) 1,1-Dimethylethyl[2-[1-(ethyloxy)ethenyl]-5-fluoro-6-(methyloxy)-3-pyridinyl]carbamate

A degassed solution of 1,1-dimethylethyl[2-chloro-5-fluoro-6-(methyloxy)-3-pyridinyl]carbamate (20 g, 72.2 mmol) in 1,4-dioxane (200 ml) was treated with bis(tri-tert-butylphosphine)palladium(0) (1.05 g), caesium fluoride (21.93 g) and tributyl[1-(ethyloxy)ethenyl]stannane (26.9 ml) then heated to 100° C. overnight. The cooled mixture was treated with 10% aqueous potassium fluoride solution. After 0.5 hour stirring, the mixture was filtered through Kieselguhr, washing with 1,4-dioxane. Ethyl acetate and water were added to the filtrate. The phases were separated and the aqueous phase extracted twice with ethyl acetate. The combined organic extracts were dried over magnesium sulphate and evaporated. The residue was rapidly chromatographed eluting with 25-50% dichloromethane in hexane affording the product as a light brown oil (22.2 g, 99%).

MS (+ve ion electrospray) m/z 313 (MH+).

### (d) 1,1-Dimethylethyl[5-fluoro-2-(fluoroacetyl)-6-(methyloxy)-3-pyridinyl]carbamate

A mixture of 1,1-dimethylethyl[2-[1-(ethyloxy)ethenyl]-5-fluoro-6-(methyloxy)-3-pyridinyl]carbamate (6.72 g, 21.54 mmol), acetonitrile (70 ml), and saturated aqueous sodium bicarbonate solution (15 ml) was treated portionwise over 5 minutes at 0° C. under argon with [1-(chloromethyl)-4-fluoro-1,4-diazoniabicyclo[2.2.2]octane bis(tetrafluoroborate) (Selectfluor™) (8.9 g, 25.05 mmol). After the addition the mixture was stirred at room temperature for 0.5 hour then treated with saturated aqueous sodium bicarbonate solution (50 ml), stirred for 10 minutes, then diluted with water (100 ml) and extracted with ethyl acetate (3×200 ml). The combined organic extracts were dried over magnesium sulphate and evaporated. The residue was chromatographed eluting with 0-50% dichloromethane in hexane affording the product as a white solid (3.72 g, 57%).

MS (+ve ion electrospray) m/z 247 (M(-t-Bu)H+).

### (e) 1,1-Dimethylethyl[2-[3-(dimethylamino)-2-fluoroacryloyl]-5-fluoro-6-(methyloxy)-3-pyridinyl]carbamate

A solution of 1,1-dimethylethyl[5-fluoro-2-(fluoroacetyl)-6-(methyloxy)-3-pyridinyl]carbamate (3.72 g, 12.32 mmol) in dry toluene (20 ml) was treated with {(dimethylamino)[(1,1-dimethylethyl)oxy]methyl}dimethylamine (Bredereck's reagent) (3.05 ml, 14.78 mmol) and heated at 40° C. for 2 hours. The mixture was evaporated and the residue triturated with 3×200 ml hexane and the remaining solid dried in vacuo to afford the product (2.15 g, 49%).

MS (+ve ion electrospray) m/z 358 (MH+).

### (f) 8-Bromo-3,7-difluoro-2-(methyloxy)-1,5-naphthyridine

A solution of 1,1-dimethylethyl[2-[3-(dimethylamino)-2-fluoroacryloyl]-5-fluoro-6-(methyloxy)-3-pyridinyl]carbamate (2.15 g, 6.02 mmol) in trifluoroacetic acid/dichloromethane (20 ml/20 ml) was stirred at room temperature for 0.5 hour under argon and evaporated. The residue was treated with ˜50 ml2M ammonia in methanol until basic and evaporated. This residue was dried in vacuo then chromatographed eluting with 0-10% methanol in ethyl acetate affording a solid (3.64 g), 8-hydroxy-3,7-difluoro-2-(methyloxy)-1,5-naphthyridine. 8-Hydroxy-3,7-difluoro-2-(methyloxy)-1,5-naphthyridine (3.64 g) was dissolved in N,N-dimethylformamide (20 ml) and treated with phosphorus tribromide (0.68 ml, 7.22 mmol). After 1 hour, more phosphorus tribromide (0.68 ml, 7.22 mmol) was added. After 0.5 hour the mixture was diluted with water and basified with solid potassium carbonate. Filtration and drying in vacuo afforded the product (0.95 g, 57%).

MS (+ve ion electrospray) m/z 275/277 (MH+).

### (g) 3,7-Difluoro-8-(1-methylethenyl)-2-(methyloxy)-1,5-naphthyridine

A solution of 8-bromo-3,7-difluoro-2-(methyloxy)-1,5-naphthyridine (1.14 g, 4.15 mmol) and tetrakis(triphenylphosphine)palladium(0) (240 mg, 0.21 mmol) in degassed dimethoxyethane (40 ml) was stirred under argon for 30 minutes. Potassium carbonate (570 mg, 4.2 mmol), water (12 ml) and pyridine - tris(1-methylethenyl)boroxin (1:1) (470 mg, 1.66 mmol) were added the mixture was heated to reflux for 5 hours. The cooled mixture was treated with water (100 ml) and ether (200 ml). The phases were separated and the aqueous phase further extracted with ether (2×200 ml). The combined organic extracts were dried over magnesium sulphate and evaporated. The residue was chromatographed on silica eluting with a gradient of 0-50% ethyl acetate in hexane affording a white solid (820 mg, 84%).

MS (+ve ion electrospray) m/z 237 (MH+).

### (h) 8-[1-(Chloromethyl)ethenyl]-3,7-difluoro-2-(methyloxy)-1,5-naphthyridine

A solution of 3,7-difluoro-8-(1-methylethenyl)-2-(methyloxy)-1,5-naphthyridine (820 mg, 3.5 mmol) in tent-butanol (50 ml) under argon was treated with cerium(III) chloride heptahydrate (1.29 g, 3.5 mmol) followed by a solution of sodium hypochlorite (12% w/v, 2.6 ml, 4.2 mmol). After 30 minutes stirring, saturated aqueous sodium sulphite solution (20 ml) was added. After 15 minutes stirring the mixture was extracted with ether (3×200 ml). The combined organic extracts were dried over magnesium sulphate and evaporated. The residue was chromatographed on silica eluting a gradient of 0-50% ethyl acetate in hexane affording a white solid (320 mg, 34%).

MS (+ve ion electrospray) m/z 271/273 (MH+).

### (i) 3,8-Difluoro-4-methylidene-4,5-dihydro-7H-pyrrolo[3,2,1-de]-1,5-naphthyridin-7-one

A mixture of 8-[1-(chloromethyl)ethenyl]-3,7-difluoro-2-(methyloxy)-1,5-naphthyridine (320 mg, 1.18 mmol) and sodium iodide (885 mg) in acetonitrile (20 ml) was heated to reflux for 20 hours. The cooled mixture was evaporated then diluted with water and extracted with dichloromethane. The organic extracts were dried over magnesium sulphate and evaporated. The residue was chromatographed on silica eluting with a gradient of 0-100% ethyl acetate in hexane affording product (168 mg, 65%).

MS (+ve ion electrospray) m/z 221 (MH+).

### (j) 3,8-Difluoro-4-hydroxy-4-(hydroxymethyl)-4,5-dihydro-7H-pyrrolo[3,2,1-de]-1,5-naphthyridin-7-one

A solution of 3,8-difluoro-4-methylidene-4,5-dihydro-7H-pyrrolo[3,2,1-de]-1,5-naphthyridin-7-one (631 mg, 2.9 mmol) in tert-butanol/water (30 ml/30 ml) was treated with AD mix alpha (4.3 g) under argon. After 18 hours stirring the reaction mixture was treated with saturated sodium sulphite solution (30 ml). After 10 minutes stirring the mixture was extracted with 20% methanol in dichloromethane (3×300 ml). The organic extracts were dried over magnesium sulphate and evaporated affording a white solid (710 mg, 97%).

MS (+ve ion electrospray) m/z 255 (MH+).

### (k) [3,8-difluoro-4-hydroxy-7-oxo-4,5-dihydro-7H-pyrrolo[3,2,1-de]-1,5-naphthyridin-4-yl]methyl 4-methylbenzenesulfonate

A solution of 3,8-difluoro-4-hydroxy-4-(hydroxymethyl)-4,5-dihydro-7H-pyrrolo[3,2,1-de]-1,5-naphthyridin-7-one (710 mg, 2.79 mmol) in dichloromethane (35 ml) and tetrahydrofuran (35 ml) was treated with triethylamine (0.58 ml, 4.19 mmol), para-toluenesulphonyl chloride (586 mg, 3.07 mmol) and dibutyltin oxide (35 mg, 0.14 mmol). After 6 hours stirring at room temperature under argon, the mixture was evaporated and water (200 ml) was added. The aqueous phase was extracted with dichloromethane (3×200 ml) and the combined organic extracts dried over magnesium sulphate and evaporated to give the desired compound (1.2 g, 100%) which was used without further purification.

MS (+ve ion electrospray) m/z 409 (MH+).

### (l) 1,1-dimethylethyl(1-{[3,8-difluoro-4-hydroxy-7-oxo-4,5-dihydro-7H-pyrrolo[3,2,1-de]-1,5-naphthyridin-4-yl]methyl}-4-piperidinyl)carbamate

A solution of crude [3,8-difluoro-4-hydroxy-7-oxo-4,5-dihydro-7H-pyrrolo[3,2,1-de]-1,5-naphthyridin-4-yl]methyl 4-methylbenzenesulfonate (1.2 g, 2.79 mmol) in ethanol (40 mL) was treated with sodium carbonate (889 mg, 8.38 mmol) and 1,1-dimethylethyl 4-piperidinylcarbamate (615 mg, 3.07 mmol). The reaction mixture was stirred at room temperature for 18 hours under argon, evaporated and treated with water (200 mL). The aqueous layer was extracted with a 10% solution of methanol in dichloromethane (3×200 mL) and the combined organic extracts dried over magnesium sulphate and evaporated. The residue was chromatographed eluting with a 0-10% gradient of methanol in dichloromethane affording a yellow solid (1.1 g, 91%).

MS (+ve ion electrospray) m/z 437 (MH+).

### (m) Title Compound

To a solution of 1,1-dimethylethyl(1-{[3,8-difluoro-4-hydroxy-7-oxo-4,5-dihydro-7H-pyrrolo[3,2,1-de]-1,5-naphthyridin-4-yl]methyl}-4-piperidinyl)carbamate (1.11 g, 2.5 mmol) in chloroform (20 mL) was added a 4M solution of HCl in 1,4-dioxane (20 mL). The reaction mixture was stirred for 15 min then methanol (20 mL) was added and stirred for 15 min, evaporated and redissolved in water (40 mL). Sodium carbonate (˜1 g) was added until basic. The aqueous layer was extracted with a 20% solution of methanol in dichloromethane (4×200 mL). The combined organic extracts were dried over magnesium sulphate and evaporated affording the crude product (864 mg, 100%).

MS (+ve ion electrospray) m/z 337 (MH+).

A portion of this material (740 mg, estimated ratio of E1:E2 ca. 3:1) was resolved by preparative chiral HPLC into the two enantiomers E1 and E2, using a Chiralpak AD column, eluting with 50:50:0.1- CH₃CN:isopropylalcohol:isopropylamine affording 553 mg of the E1 (4S) enantiomer (>99.5% ee) as the first eluting enantiomer.

### Preparation 10 (4R/S)-8-Chloro-4-({4-amino-1-piperidinyl}methyl)-3-fluoro-4-hydroxy-4,5-dihydro-7H-pyrrolo[3,2,1-de]-1,5-naphthyridin-7-one

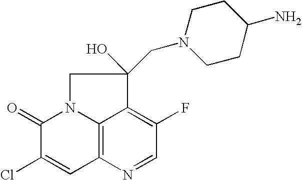

### (a) (4R/S)-8-Chloro-3-fluoro-4-hydroxy-4-(hydroxymethyl)-4,5-dihydro-7H-pyrrolo[3,2,1-de]-1,5-naphthyridin-7-one

A mixture of (4R/S)-3-fluoro-4-hydroxy-4-(hydroxymethyl)-4,5-dihydro-7H-pyrrolo[3,2,1-de]-1,5-naphthyridin-7-one (1.44 g, 6.1 mmol) and N-chlorosuccinimide (2.04 g, 15.25 mmol) in acetic acid (40 mL) and water (25 mL) was heated at 100° C. for 1 hour. An additional 2.5 equivalent of N-chlorosuccinimide (2.04 g, 15.25 mmol) was added and the rection mixture was stirred for a further hour, evaporated, treated with an aqueous solution of sodium carbonate (100 mL) and extracted with a 10% solution of methanol in dichloromethane (3×500 mL). The aqueous layer was reduced to ˜20 mL and extracted further with a 20% solution of methanol in dichloromethane (3×500 mL). The combined organic layers were dried over magnesium sulphate and evaporated affording the product with extra salts (2.2 g, 130%).

MS (+ve ion electrospray) m/z 271/273 (MH+).

### (b) ((4R/S)-8-Chloro-3-fluoro-4-hydroxy-7-oxo-4,5-dihydro-7H-pyrrolo[3,2,1-de]-1,5-naphthyridin-4-yl)methyl 4-methylbenzenesulfonate

To a suspension of impure (4R/S)-8-chloro-3-fluoro-4-hydroxy-4-(hydroxymethyl)-4,5-dihydro-7H-pyrrolo[3,2,1-de]-1,5-naphthyridin-7-one (2.20 g, assumed 6.10 mmol) in dichloromethane/tetrahydrofuran/N,N-dimethylformamide (50 mL/50 mL/5 mL) was added para-toluenesulphonyl chloride (1.16 g, 6.10 mmol), dibutyltin oxide (76 mg, 0.31 mmol) and finally triethylamine (1.27 mL, 9.15 mmol). The reaction mixture was stirred at room temperature, under argon, for 3 hours, treated with water (100 mL) and extracted with dichloromethane (3×200 mL). The combined organic layers were dried over magnesium sulphate, evaporated and further dried in vacuo affording the crude product (46%), epoxide (8′-chloro-3′-fluoro-7′H-spiro[oxirane-2,4′-pyrrolo[3,2,1-de][1,5]naphthyridin]-7′-one) (10%) and unknown material (30%).

MS (+ve ion electrospray) m/z 425/427 (MH+).

### (c) 1,1-dimethylethyl{1-[((4R/S)-8-chloro-3-fluoro-4-hydroxy-7-oxo-4,5-dihydro-7H- pyrrolo[3 ,2,1-de]-1,5-naphthyridin-4-yl)methyl]-4-piperidinyl}carbamate

To a solution of crude ((4R/S)-8-chloro-3-fluoro-4-hydroxy-7-oxo-4,5-dihydro-7H-pyrrolo[3,2,1-de]-1,5-naphthyridin-4-yl)methyl 4-methylbenzenesulfonate (˜2.5 g, assumed 6.10 mmol) in ethanol (50 mL), at room temperature, under argon was added 1,1-dimethylethyl 4-piperidinylcarbamate (1.22 g, 6.10 mmol) and sodium hydrogenocarbonate (1.94 g, 18.31 mmol). The reaction mixture was stirred for 8 hours. A further 0.5 equivalent of 1,1-dimethylethyl 4-piperidinylcarbamate (0.61 g, 3.05 mmol) and 1.5 equivalent of sodium hydrogenocarbonate (0.97 g, 9.15 mmol) were added. The reaction mixture was stirred for an additional 18 hours, treated with water (200 mL) and extracted with a 10% solution of methanol in dichloromethane (3×200 mL). The combined organic layers were dried over magnesium sulphate, evaporated and chromatograped affording an impure material which used crude in the following step.

### (d) Title Compound

A solution of crude 1,1-dimethylethyl{1-[((4R/S)-8-chloro-3-fluoro-4-hydroxy-7-oxo-4,5-dihydro-7H-pyrrolo[3,2,1-de]-1,5-naphthyridin-4-yl)methyl]-4-piperidinyl}carbamate (-1 g, 2 mmol) in dichloromethane/methanol (20 mL/20 mL) was treated with a 4M solution of HCl in 1,4-dioxane ((20 mL) was stirred at room temperature, under argon, for 30 min. The reaction mixture was evaporated, dissolved in water (˜10 mL), basified by addition of solid sodium carbonate and evaporated. The residue was stirred with a 15% solution of methanol in dichloromethane (3×200 mL). The combined organic layers were dried over magnesium sulphate, evaporated and chromatographed eluting with a gradient of dichloromethane and 2M ammonia/methanol affording the product (215 mg, 27%).

MS (+ve ion electrospray) m/z 353/355 (MH+).

### Preparation 11 (4R/S)-4-({4-amino-1-piperidinyl}methyl)-3,4-difluoro-4,5-dihydro-7H-pyrrolo[3,2,1-de]-1,5-naphthyridin-7-one dihydrochloride

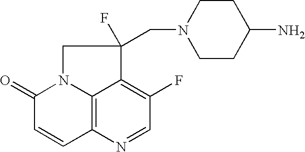

### (a) 1,1-Dimethylethyl{1-[((4R/S)-3,4-difluoro-7-oxo-4,5-dihydro-7H-pyrrolo[3,2,1-de]-1,5-naphthyridin-4-yl)methyl]-4-piperidinyl}carbamate

A solution of 1,1-dimethylethyl{1-[(3-fluoro-4-hydroxy-7-oxo-4,5-dihydro-7H-pyrrolo[3,2,1-de]-1,5-naphthyridin-4-yl)methyl]-4-piperidinyl}carbamate (15.93 g, 38.1 mmol) in dichloromethane (320 ml) was cooled in an ice bath and treated with (diethylamino)sulphur trifluoride (10.1 ml, 75.9 mmol). After 2.5 hours at room temperature saturated aqueous sodium bicarbonate solution was added, then the phases separated. The aqueous phase was extracted twice more with dichloromethane and the combined organic extracts combined and evaporated. Chromatography eluting with a methanol in dichloromethane gradient afforded the product (5.35 g, 33%).

MS (+ve ion electrospray) m/z 421 (MH+).

### (b) Title Compound

A solution of 1,1-dimethylethyl{1-[((4R/S)-3,4-difluoro-7-oxo-4,5-dihydro-7H-pyrrolo[3,2,1-de]-1,5-naphthyridin-4-yl)methyl]-4-piperidinyl}carbamate (98 mg, 0.23 mmol) in dichloromethane (2 ml) and methanol (1.5 ml), was treated with hydrochloric acid in 1,4-dioxane (4M, 3.5 ml, 14 mmol). After 1.5 hours the mixture was evaporated affording the product (82 mg).

MS (+ve ion electrospray) m/z 321 (MH+).

### Preparation 12 4-({4-amino-1-piperidinyl}methyl)-3,4-difluoro-4,5-dihydro-7H-pyrrolo[3,2,1-de]-1,5-naphthyridin-7-one (E1 and E2 enantiomer)

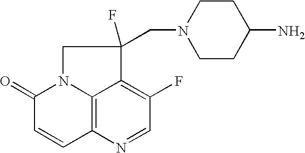

4.3 g of the free base of (4R/S)-4-[(4-amino-1-piperidinyl)methyl]-3,4-difluoro-4,5-dihydro-7H-pyrrolo[3,2,1-de]-1,5-naphthyridin-7-one dihydrochloride was resolved by preparative chiral HPLC into the two enantiomers E1 and E2 using a Chiralpak AD column, eluting with 50:50:0.1 acetonitrile:methanol:isopropylamine, affording enantiomers E1 (first eluting) and E2 (second eluting).

E1 net: 1.5 g, >99% ee

E2 net: 2.0 g, >99% ee

### Preparation 13 (4S)-4-[(4-amino-1-piperidinyl)methyl]-4-hydroxy-3-(methyloxy)-4,5-dihydro-7H-pyrrolo[3,2,1-de]-1,5-naphthyridin-7-one enantiomer E1

(4S)-4-[(4-Amino-1-piperidinyl)methyl]-3-fluoro-4-hydroxy-4,5-dihydro-7H-pyrrolo[3,2,1-de]-1,5-naphthyridin-7-one, (enantiomer E1) was converted to its corresponding formate salt (0.72 g, 2 mmol), dissolved in methanol (7 mL) and then treated with sodium methoxide (25% in methanol, 1.3 mL, 6 mmol). The reaction was then heated at reflux under argon for approx. 2 h. The cooled mixture was treated with approx. 12 drops of aqueous ammonium chloride and evaporated to dryness. The residue was extracted twice with 20% methanol/dichloromethane and the extracts were filtered and evaporated to give the product (0.66 g, 100%).

MS (+ve ion electrospray) m/z 331 (MH+).

### Preparation 14 (2E)-3-(2,5-difluorophenyl)-2-propenal

### (a) Methyl(2E)-3-(2,5-difluorophenyl)-2-propenoate

To a suspension of trans-2,5-difluorocinnamic acid in methanol at 0° C. was added thionyl chloride and the mixture was stirred at room temperature overnight. The reaction mixture was evaporated and the product purified on silica gel column and eluted with hexane/ethyl acetate gradient 0-80% to give 297 mg of white solid.

### (b) (2E)-3-(2,5-difluorophenyl)-2-propen-1-ol

To a solution of methyl(2E)-3-(2,5-difluorophenyl)-2-propenoate in DCM (6 ml) at −78° C. under N₂ was added DIBAL-H 1.5M toluene (2.21 ml, 3.22 mmol). The mixture was stirred at −78° C. for 30 min. Further DIBAL-H 1.5M (0.3 ml) was added and after 10 min at the same temperature, the reaction was completed. A mixture of methanol/acetic acid (2 ml/1 ml) and water was added to the reaction mixture. The layers were separated and the organic layer was washed with saturated NaCl, dried over Na₂SO₄, filtered and evaporated under reduced pressure to give 116 mg of desired product.

### (c) Title Compound

To a solution of (2E)-3-(2,5-difluorophenyl)-2-propen-1-ol (116 mg, 0.68 mmol) was added MnO₂ (474 mg, 5.45 mmol) and the mixture was stirred at room temperature overnight. The reaction mixture was filtered and it was evaporated to give 93 mg of white solid.

### Preparation 15 (2E)-3-(3-fluorophenyl)-2-propenal

### (a) (2E)-3-(3-fluorophenyl)-2-propenoate

To a suspension of (2E)-3-(3-fluorophenyl)-2-propenoic acid (840 mg, 1 eq) in methanol (8 ml) at 0° C. was added thionyl chloride (0.443 ml, 1.2 eq) dropwise. The reaction mixture was stirred at rt overnight. The reaction mixture was concentrated under reduced pressure to give 904 mg of product as a yellow liquid.

MS (ES+) m/z 181 (MH+).

### (b) (2E)-3-(3-fluorophenyl)-2-propen-1-ol

To a solution of methyl(2E)-3-(3-fluorophenyl)-2-propenoate in DCM (15 ml) stirred under nitrogen at 0° C. was added a solution of DIBAL-H 1.5 M in toluene dropwise. The reaction mixture was stirred at 0° C. for 3 hour. A solution of Na,K Tartrate saturated in water was added, and then it was extracted 3 times with DCM. The combined organic phases were washed with saturated NaCl, dried over anhydrous Na₂SO₄ and concentrated to give 711 mg of title compound as a colorless liquid that was used in next step without further purification.

### (c) Title Compound

Manganese dioxide (2.844 g, 32.7 mmol) was added to a solution of (2E)-3-(3-fluorophenyl)-2-propen-1-ol (711 mg, 4.67 mmol) in DCM (30 ml) at rt and the mixture was stirred at that temperature overnight. More manganese dioxide (2.844 g, 32.7 mmol) was added until TLC showed full conversion. The solids were filtered off and the solvent evaporated, yielding title compound (587.7 mg, 3.91 mmol, 84% yield) N4890-39-1 as a pale yellow oil.

MS (ES+) m/z 151 (MH+).

### Preparation 16 Example 8. 3-(3-fluorophenyl)propanal

### (a) 3-(3-fluorophenyl)-1-propanol

To a solution of methyl(2E)-3-(3-fluorophenyl)-2-propenoate (for a synthesis see Preparation 15(a)) in DCM (15 ml) stirred under nitrogen at 0° C. was added a solution of DIBAL-H in toluene 1.5 M dropwise. The reaction mixture was stirred at −78° C. for 3 hour. A Na,K Tartrate saturated solution in water was added dropwise, and then it was extracted 3 times with DCM. The combined organic phases were washed with saturated NaCl, dried over anhydrous Na₂SO₄ and concentrated to give 626.4 mg of product that was used in next step without further purification.

### (b) Title Compound

Dimethyl sulfoxide (333 μA, 4.69 mmol) was added to a solution of (ClCO)₂ (1.004 ml, 2.008 mmol) in DCM (7 ml) at −78° C. After 5 min, a solution of 3-(3-fluorophenyl)-1-propanol (206.5 mg, 1.339 mmol) in DCM (4 ml) was added dropwise and stirred for 15 min at −78° C. Triethylamine (784 μA, 5.62 mmol) was slowly added and the mixture was stirred at −78° C. for 10 min and then allowed to attain rt and stirred for an additional hour. Water and t-butylmethylether were added. Extraction, drying (MgSO₄), filtration, and concentration afforded 242.1 mg of crude product. Purification by chromatography using a 5 g silica gel cartridge, and mixtures of dichloromethane and hexane as eluant afforded 135.4 mg of title compound.

### Preparation 17 (2E)-3-(5-fluoro-3-pyridinyl)-2-propenal

A mixture of 3-bromo-5-fluoropyridine (816 mg, 4.64 mmol), trans-Di(t-acetato)bis[o-(di-o-tolyl-phosphino)benzyl]dipalladium(II) (87 mg, 0.093 mmol) and sodium acetate (423 mg, 5.16 mmol) was introduced in a pressure tube under argon. Then NMP (12 ml) previously degassed and then 2-propenal (684 μA, 4.64 mmol) was consecutively added to the reaction mixture. After 2 h at 140° C. argon atmosphere the reaction was completed. H₂O and DCM was added and the organic layer was separated, washed with brine, dried over Na₂SO₄ and solvent removed by vacuum. NMP was removed under vacuum to give 425 mg of a brown oil. The crude product was purified by silica chromatography (10 g Si, Hexane:ethyl acetate 8:2) to give 87 mg of a pale solid.

MS (ES+) m/z 152 [M+H]⁺

### Preparation 18 [(3-fluorophenyl)oxy]acetaldehyde

### (a) Methyl[(3-fluorophenyl)oxy]acetate

To a solution of [(3-fluorophenyl)oxy]acetic acid (200 mg, 1.175 mmol) in methanol (3 ml) was added thionyl chloride (0.086 ml, 1.175 mmol) and the mixture was stirred at room temperature for 3 h. The reaction mixture was evaporated to dryness to give 203 mg of colorless oil.

### (b) Title Compound

To a solution of methyl[(3-fluorophenyl)oxy]acetate (200 mg, 1.086 mmol) in DCM (6 ml) at −78° C. under N₂ was added a solution of DIBAL-H (1.140 ml of 1M solution in THF, 1.140 mmol). The mixture was stirred at −78° C. After 1 h at −78° C. 0.76 mL of DIBAL-H (1.5 M solution in toluene, 1.140 mmol) were added to the reaction mixture and then stirring was continued at −78° C. for 1.5 h. As the conversion of the reaction was not complete 0.760 ml DIBAL-H (1.5 M solution in toluene 1.140 mmol) was added and the mixture finally stirred for an addition 1 h at −78° C. The reaction mixture was diluted with DCM, Rochelle salt (potassium sodium tartrate tetrahydrate) solution in water was added. The layers were separated and the organic layer was dried over Na₂SO₄, filtered and evaporated under reduced pressure to give 144 mg of colorless oil.

### Biological Activity

### Antimicrobial Activity Assay:

Whole-cell antimicrobial activity was determined by broth microdilution using the Clinical and Laboratory Standards Institute (CLSI) recommended procedure, Document M7-A7, “Methods for Dilution Susceptibility Tests for Bacteria that Grow Aerobically”. The compounds were tested in serial two-fold dilutions ranging from 0.016 to 16 mcg/mL.

The minimum inhibitory concentration (MIC) was determined as the lowest concentration of compound that inhibited visible growth. A mirror reader was used to assist in determining the MIC endpoint.

Compounds were evaluated against Gram-positive organisms, including Staphylococcus aureus, Streptococcus pneumoniae, Streptococcus pyogenes, Enterococcus faecalis and Enterococcus faecium.

In addition, compounds were evaluated against Gram-negative strains including Haemophilus influenzae, Moraxella catarrhalis, Escherichia coli, Pseudomonas aeruginosa, Proteus mirabilis, Enterobacter cloacae, Enterobacter aerogenes, Klebsiella pneumoniae and Stenotrophomonas maltophilia.

Examples 1, 2 and 4-10, as identified in the present application, were tested in at least one exemplified salt form, and had a MIC ≦2 μg/ml against a strain of at least one of the organisms listed above. For at least one strain of every organism listed above, at least one tested Example had a MIC ≦2 μg/ml.

Mycobacterium tuberculosis H37Rv Inhibition Assay

The measurement of the minimum inhibitory concentration (MIC) for each tested compound was performed in 96 wells flat-bottom, polystyrene microtiter plates. Ten two-fold drug dilutions in neat DMSO starting at 400 μM were performed. Five μl of these drug solutions were added to 95 μl of Middlebrook 7H9 medium. (Lines A-H, rows 1-10 of the plate layout). Isoniazid was used as a positive control, 8 two-fold dilution of Isoniazid starting at 160 μgml⁻¹ was prepared and 5 μl of this control curve was added to 95 μl of Middlebrook 7H9 (Difco catalogue Ref. 271310)+ADC medium (Becton Dickinson Catalogue Ref 211887). (Row 11, lines A-H). Five μl of neat DMSO were added to row 12 (growth and Blank controls).

The inoculum was standardised to approximately 1×10⁷ cfu/ml and diluted 1 in 100 in Middlebrook 7H9+ADC medium and 0.025% Tween 80 (Sigma P4780), to produce the final inoculum of H37Rv strain (ATCC25618). One hundred μl of this inoculum was added to the entire plate but G-12 and H-12 wells (Blank controls). All plates were placed in a sealed box to prevent drying out of the peripheral wells and they were incubated at 37° C. without shaking for six days. A resazurin solution was prepared by dissolving one tablet of resazurin (Resazurin Tablets for Milk Testing; Ref 330884Y VWR International Ltd) in 30 ml sterile PBS (phosphate buffered saline). 25 μl of this solution was added to each well. Fluorescence was measured (Spectramax M5 Molecular Devices, Excitation 530 nm, Emission 590 nm) after 48 hours to determine the MIC value. Examples 1 and 2 were tested in the Mycobacterium tuberculosis H37Rv inhibition assay. Examples 1 and 2 showed an MIC value of 5.1 μg/ml or lower. Example 1 showed an MIC value of 1.0 μg/ml.

1. A compound of formula (I) or a pharmaceutically acceptable salt thereof: 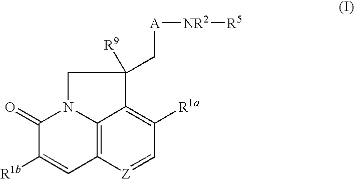
wherein:
Z is CH or N;
R^{1a} and R^{1b} are independently selected from hydrogen; halogen; cyano; (C₁₋₆)alkyl; (C₁₋₆)alkylthio; trifluoromethyl; trifluoromethoxy; carboxy; hydroxy optionally substituted with (C₁₋₆)alkyl or (C₁₋₆)alkoxy-substituted(C_{1 -6})alkyl; (C_{1 -6})alkoxy-substituted(C₁₋₆)alkyl; hydroxy (C₁₋₆)alkyl; an amino group optionally N-substituted by one or two (C₁₋₆)alkyl, formyl, (C₁₋₆)alkylcarbonyl or (C₁₋₆)alkylsulphonyl groups; and aminocarbonyl wherein the amino group is optionally substituted by (C₁₋₄)alkyl;
R² is hydrogen, or (C₁₋₄)alkyl, or together with R⁶ forms Y as defined below;
A is a group (ia) or (ib): 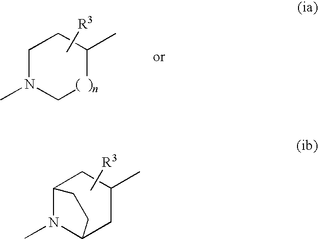
in which: R³ is as defined for R^{1a} or R^{1b} or is oxo and n is 1 or 2:
or A is a group (ii) 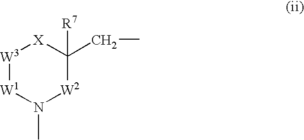
W¹, W² and W³ are CR⁴R⁸
or W² and W³ are CR⁴R⁸ and W¹ represents a bond between W³ and N.
X is O, CR⁴R⁸, or NR⁶;
one R⁴ is as defined for R^{1a} and R^{1b} and the remainder and R⁸ are hydrogen or one R⁴ and R⁸ are together oxo and the remainder are hydrogen;
R⁶ is hydrogen or (C₁₋₆)alkyl; or together with R² forms Y;
R⁷ is hydrogen; halogen; hydroxy optionally substituted with (C₁₋₆)alkyl; or (C₁₋₆alkyl;
Y is CR⁴R⁸CH₂; CH₂CR⁴R⁸; (C═O); CR⁴R⁸; CR⁴R⁸(C═O); or (C═O)CR⁴R⁸;
or when X is CR⁴R⁸, R⁸ and R⁷ together represent a bond;
R⁵ is a group —X^{1a}—X^{2a}—X^{3a}—X^{4a} in which:
X^{1a} is CH₂, CO or SO₂;
X^{2a} is CR^{14a}R^{15a};
X^{3a} is NR^{13a}; O, S, SO₂ or CR^{14a}R^{15a}; wherein:
each of R^{14a} and R^{15a} is independently selected from: H; (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxyl ; (C_{1- 4})alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)allcenyl; aryl; aryl(C₁₋₄)alkyl; aryl(C₁₋₄)alkoxy; provided that R¹⁴a and R¹⁵a ₒₙ the same carbon atom are not both selected from optionally substituted hydroxy and optionally substituted amino; or
R^{14a} and R^{15a} together represent oxo;
R^{13a} is hydrogen; trifluoromethyl; (C₁₋₆)allcyl; (C₂₋₆)alkenyl; (C₁₋₆)alkoxycarbonyl; (C₁₋₆)alkylcarbonyl; or aminocarbonyl wherein the amino group is optionally substituted by (C₁₋₆)alkoxycarbonyl, (C₁₋₆)alkylcarbonyl, (C₂₋₆)alkenyloxycarbonyl, (C₂₋₆)alkenylcarbonyl, (C₁₋₆)alkyl or (C₂₋₆)alkenyl and optionally further substituted by (C₁₋₆)alkyl or (C₂₋₆)alkenyl; or
two R^{14a} groups or an R^{13a} and an R^{14a} group on adjacent atoms together represent a bond and the remaining R^{13a}, R^{14a} and R^{15a} groups are as above defined; or
two R^{14a} groups and two R^{15a} groups on adjacent atoms together represent bonds such that X^{2a} and X^{3a} is triple bonded;
X^{4a} is phenyl or C or N linked monocyclic aromatic 5- or 6-membered heterocycle containing up to four heteroatoms selected from O, S and N and: optionally C-substituted by up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)allcoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted as for corresponding substituents in R³; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; aryl, aryl(C₁₋₄)alkyl or aryl(C₁₋₄)allcoxy; and
optionally N substituted by trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl(C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl; and
R⁹ is hydrogen or hydroxy, or when Z is N R⁹ may instead be fluoro.

2. A compound according to claim 1 wherein Z is CH and R⁹ is hydrogen and the stereochemistry at the carbon atom to which the group R⁹ is attached is R, or Z is N and R⁹ is OH and the stereochemistry at the carbon atom to which the group R⁹ is attached is S.

3. A compound according to any preceding claim 1 wherein R^{1a} is fluoro and R^{1b} is hydrogen.

4. A compound according to any preceding claim 1 wherein R² is hydrogen.

5. A compound according to any preceding claim 1 wherein A is (ia), n is 1 and R³ is H or hydroxy in the 3-position, A is (ii), X is CR⁴R⁸ and R⁸ is H and R⁴ is H or OH, or A is (ii), X is O, R⁷ is H and W¹, W² and W³ are each CH₂.

6. A compound according to claim 4 wherein A is piperidin-4-yl or pyrrolidin-3-ylmethyl.

7. A compound according to claim 1 wherein —X^{1a}—X^{2a}—X^{3a}— is —(CH₂)₂—O—, —CH₂—CH═CH—, —(CH₂)₃—, —(CH₂)₂—NH— or —CH₂CONH—.

8. A compound according to any claim 1 wherein X^{4a} is pyrid-2-yl, pyrid-3-yl, thiazole-2-yl, pyrimidin-2-yl, pyrimidin-5-yl or fur-2-yl, optionally substituted by halo, trifluoromethyl or nitro.

9. A compound according to claim 1 wherein X^{4a} is phenyl optinally substituted by halo, nitro, cyano, trifluoromethyl, methyl, 1-methylethyl, methoxycarbonyl and methylcarbonylamino.

10. A compound according to claim 1 wherein X^{4a} is pyrid-2-yl, fur-2-yl, 4-(1-methylethyl)phenyl, pyrid-3-yl, 2,5-difluorophenyl, 3-fluorophenyl, 5-fluoropyrid-3-yl, 3,5-difluorophenyl or thiazol-2-yl.

11. A compound according to claim 1 selected from the group consisting of:
(4S)-3-Fluoro-4-hydroxy-4-[(4-{[(2E)-3-(2-pyridinyl)-2-propen-1-yl]amino}-1-piperidinyl)methyl]-4,5-dihydro-7H-pyrrolo[3,2,1-de]-1,5-naphthyridin-7-one;
(4S)-3-Fluoro-4-[(4-{[(2E)-3-(2-furanyl)-2-propen-1-yl]amino}-1-piperidinyl)methyl]-4-hydroxy-4,5-dihydro-7H-pyrrolo[3,2,1]-de]-1,5-naphthyridin-7-one;
9-Fluoro-1-{[4-({2-methyl-3-[4-(1-methylethyl)phenyl]propyl}amino)-1-piperidinyl]methyl}-1,2-dihydro-4H-pyrrolo[3,2,1-ij]quinolin-4-one;
(1R)-9-Fluor)-1-[(4-{[(2E)-3-(3-pyridinyl)-2-propen-1-yl]amino}-1-piperidinyl)methyl]-1,2-dihydro-4H-pyrrolo[3,2,1-if]quinolin-4-one;
(1R)-9-Fluoro-1-[(4-{[(2E)-3-(2-furanyl)-2-propen-1-yl]amino}-1-piperidinyl)methyl]-1,2-dihydro-4H-pyrrolo[3,2,1-ij]quinolin-4-one;
(1R)-1-[(4-{[(2E)-3-(2,5-Difluorophenyl)-2-propen-1-yl]amino}-1-piperidinyl)methyl]-9-fluoro-1,2-dihydro-4H-pyrrolo[3,2,1-ij]quinolin-4-one;
(1R)-9-Fluoro-1-[(4-{[(2E)-3-(3-fluorophenyl)-2-propen-1-yl]amino}-1-piperidinyl)methyl]-1,2-dihydro-4H-pyrrolo[3,2,1-ij]quinolin-4-one;
(1R)-9-Fluoro-1-[(4-{[3-(3-fluorophenyl)propyl]amino}-1-piperidinyl)methyl]-1,2-dihydro-4H-pyrrolo[3,2,1-ij]quinolin-4-one;
(1R)-9-Fluoro-1-[(4-{[(2E)-3-(5-fluoro-3-pyridinyl)-2-propen-1-yl]amino}-1-piperidinyl)methyl]-1,2-dihydro-4H-pyrrolo[3,2,1-ij]quinolin-4-one; and
(1R)-9-Fluoro-1-{[4-({2-[(3-fluorophenyl)oxy]ethyl}amino)-1-piperidinyl]methyl}-1,2-dihydro-4H-pyrrolo[3,2,1-ij]quinolin-4-one;
or a pharmaceutically acceptable salt thereof.

12. A method of treatment of bacterial infections in mammals, particularly in man, which method comprises the administration to a mammal in need of such treatment an effective amount of a compound according to claim 1.

13-15. (canceled)

16. A pharmaceutical composition comprising a compound according to claim 1 and a pharmaceutically acceptable carrier.
